SEQUENCE LISTING (13 sequences)

SEQ ID NO: 1 - protein, 449 aa, Artificial Sequence
  KappaMab VH
EVQLQQSGAELVKPGASVKLSCTASGFNIKDTYMHWVKQRPEQGLEWIGRIDPANGNTKYDPKFQGKATIIADTSSNTAYLQLSSLTSEDTAVYYCARGVYHDYDGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Artificial Sequence
  Kappamab VL
DIVMTQSQKFMSTSVGDRVSVTCKASQNVGTNVAWYQQKPGQSPKALIYSTSYRYSGVPDRFTGSGSGTDFTLTISNVQSEDLAEYFCQQYNSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 111 aa, Artificial Sequence
  Switch region Kflc
LEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 5 aa, Artificial Sequence
  KMA epitope
LDIKR

SEQ ID NO: 5 - protein, 5 aa, Artificial Sequence
  KMA epitope 2 improved binding
LEIKR

SEQ ID NO: 6 - protein, 11 aa, Artificial Sequence
  VH CDR 1
GFNIKDTYMHW

SEQ ID NO: 7 - protein, 17 aa, Artificial Sequence
  VH CDR 2
RIDPANGNTKYDPKFQG

SEQ ID NO: 8 - protein, 10 aa, Artificial Sequence
  VH CDR 3
GVYHDYDGDY

SEQ ID NO: 9 - protein, 11 aa, Artificial Sequence
  VL CDR 1
KASQNVGTNVA

SEQ ID NO: 10 - protein, 7 aa, Artificial Sequence
  VL CDR 2
STSYRYS

SEQ ID NO: 11 - protein, 9 aa, Artificial Sequence
  VL CDR 3
QQYNSYPYT

SEQ ID NO: 12 - protein, 119 aa, Artificial Sequence
  VH K121
QVQLQQSGAELVKPGASVKLSCTASGFNIKDTYMHWVKQRPEQGLEWIGRIDPANGNTKYDPKFQGKAAIIADTSSNTAYLQLSSLTSEDTAVYYCARGVYHDYDGDYWGQGTTVTVAS

SEQ ID NO: 13 - protein, 107 aa, Artificial Sequence
  VL K121
DIVMTQSQKFMSTSVGDRVSVTCKASQNVGTNVAWYQQKPGQSPKALIYSTSYRYSGVPDRFTGSGSGTDFTLTISNVQSEDLAEYFCQQYNSYPYTFGGGTKLEIK

(19) United States
(12) Patent Application Publication
(10) Pub. No.: US 2022/0031840 A1
(43) Pub. Date: Feb. 3, 2022

(54) Method of Treatment

(71) Applicant: HaemaLogix Pty Ltd, Eveleigh (AU)

(72) Inventors: Rosanne Dunn, Eveleigh (AU); Ben R. Herbert, Eveleigh (AU); Edmond J. Breen, Eveleigh (AU)

(73) Assignee: HaemaLogix Pty Ltd, Eveleigh (AU)

(21) Appl. No.: 17/309,499

(22) PCT Filed: Dec. 3, 2019
(86) PCT No.: PCT/AU2019/051317

§ 371 Date: Jun. 2, 2021

(30) Foreign Application Priority Data
Dec. 3, 2018 (AU) 2018904581

(51) Int. Cl. A61K 39/395; A61K 45/06; A61K 31/69; A61P 35/00; C07K 16/30

(52) U.S. Cl. CPC A61K 39/39558; A61K 45/06; A61K 31/69; A61P 35/00; A61K 2039/545; C07K 2317/24; C07K 2317/56; C07K 2317/565; C07K 16/3061

(57) ABSTRACT

The present disclosure relates to therapeutic combinations comprising an anti-KMA antibody and a proteasome inhibitor for the treatment of multiple myeloma. The present disclosure also relates to methods of treating multiple myeloma in subjects with high serum cytokine levels.

### FIELD OF THE INVENTION

The present disclosure relates to therapeutic combinations comprising an anti-KMA antibody and a proteasome inhibitor for the treatment of multiple myeloma. The present disclosure also relates to methods of treating multiple myeloma in subjects with high serum cytokine levels.

### BACKGROUND OF THE INVENTION

Multiple myeloma represents a malignant proliferation of plasma cells derived from a single clone. The multiple myeloma tumour, its products, and the host response to it result in a number of organ dysfunctions, symptoms of bone pain or fracture, renal failure, susceptibility to infection, anemia, hypocalcemia, clotting abnormalities, neurologic symptoms and vascular manifestations of hyperviscosity (Palumbo and Anderson 2011).

No effective long-term treatment currently exists for multiple myeloma. Systemic chemotherapy is the current front line therapy, and the current median of survival with chemotherapy is about three years.

While multiple myeloma is considered to be a drug-sensitive disease, almost all patients with multiple myeloma who initially respond to chemotherapy eventually relapse. Since the introduction of melphalan and prednisone therapy for multiple myeloma, numerous multi-drug chemotherapies including Vinca alkaloid, anthracycline, and nitrosourea-based treatment have been tested but there has been little improvement in outcome over the past three decades. Accordingly, new methods of treatment are needed.

### SUMMARY OF THE INVENTION

The present inventors have surprisingly identified that administration of an anti-KMA antibody to human subjects decreases serum concentrations of cytokines that play a significant role in the signalling pathways associated with survival of myeloma cells in the bone marrow microenvironment. Elevated levels of these cytokines have been previously linked with therapeutic resistance, in particular resistance to proteasome inhibitors in subjects with multiple myeloma. Accordingly, in one example, the present disclosure relates to a method of treating multiple myeloma in a subject in need thereof, the method comprising administering to the subject an anti-KMA antibody and a proteasome inhibitor. In another example, the present disclosure relates to a therapeutic combination comprising a proteasome inhibitor and an anti-KMA antibody, the combination being provided for simultaneous or sequential administration.

In an example, the proteasome inhibitor is selected from the group consisting of marizomib, oprozomib, epoxomicin, salinosporamide A, carfilzomib, ixazomib and bortezomib. For example, the proteasome inhibitor can be bortezomib.

In an example, the anti-KMA antibody binds to or specifically binds to an epitope of KMA that is specifically bound by kappamab or that competes with kappamab for binding to KMA, wherein kappamab has a heavy chain variable region (VH) comprising a sequence set forth in SEQ ID NO: 1 and a light chain variable region (VL) comprising a sequence set forth in SEQ ID NO: 2. In an example, the epitope of KMA comprises a sequence set forth in SEQ ID NO: 5.

In another example, the anti-KMA antibody comprises a V_{H} and a V_{L}, the V_{H} comprising a complementarity determining region (CDR) 1 comprising an amino acid sequence as shown in SEQ ID NO: 6, a CDR2 comprising an amino acid sequence as shown in SEQ ID NO: 7 and a CDR3 comprising a sequence as shown in SEQ ID NO: 8 and the V_{L} comprising a CDR 1 comprising an amino acid sequence as shown in SEQ ID NO: 9, a CDR2 comprising an amino acid sequence as shown in SEQ ID NO: 10 and a CDR3 comprising a sequence as shown in SEQ ID NO: 11. In an example, the V_{H} comprises an amino acid sequence at least about 95% identical to the amino acid sequence shown in SEQ ID NO: 1. In another example, the V_{L} comprises an amino acid sequence at least about 95% identical to the amino acid sequence shown in SEQ ID NO: 2. In another example, the V_{H} comprises an amino acid sequence as shown in SEQ ID NO: 1. In another example, the V_{L} comprises an amino acid sequence as shown in SEQ ID NO: 2. In another example, the V_{H} comprises an amino acid sequence as shown in SEQ ID NO: 1 and the V_{L} comprises an amino acid sequence as shown in SEQ ID NO: 2.

In an example, the methods of the present disclosure comprise administering the anti-KMA antibody at a dosage ranging from about 0.3 mg/kg to 30 mg/kg. In another example, the anti-KMA antibody is administered at a dosage ranging from about 1 mg/kg to 10 mg/kg. In another example, the anti-KMA antibody is administered at about 3 mg/kg. In another example, the anti-KMA antibody is administered at about 10 mg/kg. In another example, the proteasome inhibitor is administered at a dose ranging from about 0.5 mg/m² to about 1.5 mg/m².

In an example, the therapeutic combination of the present disclosure comprises anti-KMA antibody at a dosage ranging from about 0.3 mg/kg to 30 mg/kg. In another example, the anti-KMA dosage ranges from about 1 mg/kg to 3 mg/kg. In another example, the anti-KMA antibody dosage is about 3 mg/kg. In another example, the proteasome inhibitor dose ranges from about 0.5 mg/m² to about 1.5 mg/m².

In another example, the methods of the present disclosure further comprise administering one or more additional anti-cancer agents. In another example, the therapeutic combination of the present disclosure further comprises one or more additional anti-cancer agents. Exemplary additional anti-cancer agent(s) can be selected from the group consisting of a chemotherapy, an immunomodulatory drug (thalidomide, lenalidomide, pomalidomide), a histone deacetylase inhibitor (panobinostat), an antibody (elotuzumab, daratumumab, isatuximab), a steroid (dexamethasone). In an example, the additional anti-cancer agent is dexamethasone. In another example, the additional anti-cancer agents are dexamethasone and lenalidomide.

In an example, the anti-KMA antibody and proteasome inhibitor are administered simultaneously or sequentially. For example, the anti-KMA antibody and proteasome inhibitor can be administered simultaneously. In another example, the anti-KMA antibody and proteasome inhibitor can be administered sequentially.

In another example, the anti-KMA antibody is administered monthly.

Subjects treated according to the methods of the present disclosure can have failed multiple prior lines of therapy. In another example, subject has received at least one, at least two, at least three, at least four, at least five, at least six prior lines of therapy. In another example, subjects have achieved at least a minimal response (25% reduction in M protein) to their most recent line of therapy. In another example, subjects are refractory to at least one, at least two, at least three, at least four prior lines of therapy. In another example, subjects are refractory to at least one proteasome inhibitor. In this example, the proteasome inhibitor may be bortezomib.

In another example, the subjects multiple myeloma is characterised as progressive disease. In another example, the subject has relapsed myeloma. In another example, the subject has refractory myeloma. In another example, the subject has relapsed and refractory myeloma. In another example, the subject has primary refractory myeloma. In an example, the subjects myeloma is relapsed and refractory to at least a proteasome inhibitor. In an example, the subjects myeloma is relapsed and refractory to at least bortezomib.

In another example, the subjects multiple myeloma is characterised as stable disease at the time of first administration. In another example, the serum level of kappa free light chain in a sample obtained from the subject is less than about 250 mg/ml.

In an example, the serum level of HGF in a sample obtained from the subject is between about 0.3 ng/ml and 1.6 ng/ml. In another example, the serum level of MIF in a sample obtained from the subject is between about 414 pg/ml and 4707 pg/ml. In another example, the serum level of CCL27 in a sample obtained from the subject is between about 150 pg/ml and 600 pg/ml. In another example, the serum level of G-CSF in a sample obtained from the subject is between about 20 pg/ml and 65 pg/ml. In another example, the serum level of CXCL9 in a sample obtained from the subject is between about 70 pg/ml and 550 pg/ml. In another example, the serum level of CXCL10 in a sample obtained from the subject is between about 300 pg/ml and 900 pg/ml.

The methods of the present disclosure also relate to treating multiple myeloma in subjects with high serum cytokine levels. For example, the methods of the present disclosure relate to treating multiple myeloma in a subject, the method comprising selecting a subject who has high serum levels of one or more of the following factors relative to control serum levels: hepatocyte growth factor (HGF), macrophage inhibitory factor (MIF), CCL27, G-CSF, CXCL9, and CXCL10; and administering to the subject an anti-KMA antibody.

In an example, a high serum level of HGF is above about 0.5 ng/ml. In another example, a high serum level of HGF is at least about 1.6 ng/ml. In another example, a high serum level of MIF is above about 5000 pg/ml. In another example, a high serum level of CCL27 is above about 500 pg/ml. In another example, a high serum level of G-CSF is above about 55 pg/ml. In another example, a high serum level of CXCL9 is above about 550 pg/ml. In another example, a high serum level of CXCL10 is above about 850 pg/ml. High serum cytokine levels are determined in a sample obtained from the subject.

In an example, these methods further comprise administering a proteasome inhibitor. In an example, the proteasome inhibitor is selected from the group consisting of marizomib, oprozomib, epoxomicin, salinosporamide A, carfilzomib, ixazomib and bortezomib. In another example, the proteasome inhibitor is bortezomib.

In another example, the methods of the present disclosure relate to use of a proteasome inhibitor and an anti-KMA antibody defined herein in the manufacture of a medicament for the treatment of multiple myeloma. In another example, the methods of the present disclosure also relate to a proteasome inhibitor and an anti-KMA antibody defined herein for use in the treatment of multiple myeloma.

Any example herein shall be taken to apply mutatis mutandis to any other example unless specifically stated otherwise.

The present invention is not to be limited in scope by the specific examples described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying drawings.

### BRIEF DESCRIPTION OF THE ACCOMPANYING DRAWINGS

FIG. 1. Individual patient serum kappamab concentrations over time for each kappamab dosing cohort. Cohort 1=0.3 mg/kg (Panel A), Cohort 2 (Panel B)=1.0 mg/kg, Cohort 3=3.0 mg/kg (Panel C), and Cohort 4=10 mg/kg (Panel D). D, day.

FIG. 2. Patient profiles of percent change in serum κFLC concentrations from baseline after kappamab intravenous infusion, presented by dose cohort. Cohort 1 (0.3 mg/kg; Panels A and E), Cohort 2 (1.0 mg/kg; Panels B and F), Cohort 3 (3.0 mg/kg; Panels C and G), and Cohort 4 (10 mg/kg; Panels D and H). Baseline serum concentrations were assessed at about 30 minutes pre-infusion (0) and then post infusion at specified intervals up to Day 45 (Panels A-D) and during the follow-up phase for a total of 135 days (Panels E-H). FLC, free light chain.

FIG. 3. Individual patient best response after treatment with kappamab. Response expressed as percent change from baseline for serum kappa free light chain (Panel A) and M protein (Panel B) during the treatment phase (Day 45 post infusion). Best response was defined as the lowest percent increase from baseline or maximum percent decrease from baseline at the earliest time point after kappamab treatment. P, patient ID; D, day of earliest best response.

FIG. 4. Bar plots for kappamab dose effects on cytokine expression levels. Bars represent expression-adjusted means for the 6 cytokines with statistically significant observed interactions (P-value<0.05; Table 9) between cytokine expression and kappamab dose. Error bars represent 95% confidence levels. Means are adjusted according to cytokine baseline expression levels and for patient:plate effects. The Y-axis presents the log 2 of the mean cytokine fluorescence values (FI). CXCL9 (chemokine (C-X-C motif) ligand 9); HGF (hepatocyte growth factor); MIF (macrophage inhibitory factor); CXCL10 (chemokine (C-X-C motif) ligand 10; CCL27 (chemokine (C-C motif) ligand 27); G-CSF (granulocyte colony-stimulating factor).

FIG. 5. ¹⁸Fluorine-D-glucose-positron emission tomography (¹⁸FDG-PET) scans for Patient 8 (who was receiving lenalidomide) prior to kappamab treatment (Panel A) and 30 days after intravenous infusion with 3.0 mg/kg kappamab (Panel B). ¹⁸FDG-PET scanning was performed encompassing the skull vertex down to the knees on the combined PET/Computed Tomography (CT) scanner and a contemporaneous low-dose, non-contrast CT scan was performed for the purposes of attenuation correction and to provide anatomical correlation. In Panel A, multifocal, avid disease is demonstrated within the bone marrow, both within the axial and appendicular skeleton. In the spine, disease is most prominent within the sacrum. In the appendicular skeleton, disease is most pronounced within the proximal femur, where a prosthetic rod is in situ. No metabolically active disease is present outside the bones. In Panel B, ¹⁸FDG-PET scanning was performed as before to assess the response 30 days after kappamab treatment. Uptake in the previously documented multifocal bony abnormalities was largely resolved and indicated a favourable and almost complete metabolic response. Persisting uptake was seen in the medial condyle of the left femur but was less intense than the previous scan. Low-grade uptake on the right lower lateral chest wall was intense and may reflect physiological muscular activity. No abnormal uptake was seen in the lungs, liver, or spleen and no nodal uptake was detected.

### DETAILED DESCRIPTION OF THE INVENTION

### General Techniques and Selected Definitions

Unless specifically defined otherwise, all technical and scientific terms used herein shall be taken to have the same meaning as commonly understood by one of ordinary skill in the art (e.g., molecular biology, biochemistry, oncology and clinical studies).

Unless otherwise indicated, the molecular and statistical techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

One of skill in the art will be aware that an “antibody” is generally considered to be a protein that comprises a variable region made up of a plurality of polypeptide chains, e.g., a polypeptide comprising a V_{L} and a polypeptide comprising a V_{H}. An antibody also generally comprises constant domains, some of which can be arranged into a constant region or constant fragment or fragment crystallizable (Fc). A V_{H} and a V_{L} interact to form a Fv comprising an antigen binding region that specifically binds to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG1, IgG2, IgG3, IgG4, IgA1 and IgA2) or subclass. The term “antibody” also encompasses humanized antibodies, primatized antibodies, human antibodies and chimeric antibodies.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

The term “naked antibody” refers to an antibody that is not conjugated to another compound, e.g., a toxic compound or radiolabel.

An “antigen binding fragment” of an antibody comprises one or more variable regions of an intact antibody. Examples of antibody fragments include Fab, Fab′, F(ab′)2 and Fv fragments (scFv, di-scFv, tri-scFv); diabodies; linear antibodies; single-chain antibody molecules and multispecific antibodies formed from antibody fragments.

As used herein, “variable region” refers to the portions of the light and/or heavy chains of an antibody as defined herein that specifically binds to an antigen and, for example, includes amino acid sequences of CDRs; i.e., CDR1, CDR2, and CDR3, and framework regions (FRs). For example, the variable region comprises three or four FRs (e.g., FR1, FR2, FR3 and optionally FR4) together with three CDRs. V_{H} refers to the variable region of the heavy chain. V_{L} refers to the variable region of the light chain.

The term “complementarity determining regions” (syn. CDRs; i.e., CDR1, CDR2, and CDR3) is used in the context of the present disclosure to refer to the amino acid residues of an antibody variable region the presence of which are major contributors to specific antigen binding. Each variable region typically has three CDR regions identified as CDR1, CDR2 and CDR3. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”.

In another example, the amino acid positions assigned to CDRs and FRs are defined according to international ImMunoGeneTics information system, Marie-Paule Lefranc, Université de Montpellier and CNRS, 1989 (also referred to herein as “the IMGT numbering system”.

The term “EU numbering system of Kabat” will be understood to mean the numbering of an antibody heavy chain is according to the EU index as taught in Kabat et al., 1991, Sequences of Proteins of Immunological Interest, 5th Ed., United States Public Health Service, National Institutes of Health, Bethesda. The EU index is based on the residue numbering of the human IgG1 EU antibody.

“Framework regions” (Syn. FR) are those variable domain residues other than the CDR residues.

As used herein, the term “binds” in reference to the interaction of an antibody or antigen binding fragment thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labelled “A” and the antibody, will reduce the amount of labelled “A” bound to the antibody.

As used herein, the term “specifically binds” shall be taken to mean that the binding interaction between an antibody or antigen binding fragment thereof and kappa myeloma antigen is dependent on the presence of the antigenic determinant or epitope of kappa myeloma antigen bound by the antibody or antigen binding fragment thereof. Accordingly, the antibody or antigen binding fragment thereof preferentially binds or recognizes a kappa myeloma antigen determinant or epitope even when present in a mixture of other molecules or organisms. In one example, the antibody or antigen binding fragment thereof reacts or associates more frequently, more rapidly, with greater duration and/or with greater affinity with kappa myeloma antigen or a cell expressing the same than it does with alternative antigens or cells. It is also understood by reading this definition that, for example, an antibody or antigen binding fragment thereof that specifically binds to kappa myeloma antigen may or may not specifically bind to a second antigen. As such, “specific binding” does not necessarily require exclusive binding or non-detectable binding of another antigen. The term “specifically binds” can be used interchangeably with “selectively binds” herein. Generally, reference herein to binding means specific binding, and each term shall be understood to provide explicit support for the other term. Methods for determining specific binding will be apparent to the skilled person. In an example, an anti-KMA antibody according to the present disclosure is contacted with kappa myeloma antigen or a cell expressing same or a mutant form thereof or an alternative antigen. The binding of the antibody to the kappa myeloma antigen or mutant form or alternative antigen is then determined and an antibody that binds as set out above to the kappa myeloma antigen rather than the mutant or alternative antigen is considered to specifically bind to kappa myeloma antigen.

The terms “carrier” and “excipient” refer to compositions of matter that are conventionally used in the art to facilitate the storage, administration, and/or the biological activity of an active compound (see, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

Suitable carriers for the present disclosure include those conventionally used, e.g., water, saline, aqueous dextrose, lactose, Ringer's solution, a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

The term “analyte” is used in the context of the present disclosure to refer to a molecule whose presence in a sample provides a quantitative or qualitative measure of gene expression. Exemplary analytes informative of gene expression levels include RNA and protein. Various methods of determining RNA and protein levels are known in the art. Exemplary methods include whole genome sequencing, next generation sequencing, NanoString technology, droplet digital PCR, quantitative RT-PCR, mass spectrometry, immunohistochemistry and multiplex immunoassay. In an example, the analyte is a cytokine which is measured using a multiplex immunoassay (e.g. Bio-Plex Pro Human Cytokine assay; Bio-Rad).

As used in this specification and the appended claims, terms in the singular and the singular forms “a,” “an” and “the,” for example, optionally include plural referents unless the content clearly dictates otherwise.

As used herein, the term “about”, unless stated to the contrary, refers to +/−10%, more preferably +/−5%, more preferably +/−1%, of the designated value.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Therapeutic Combination

In one example, the present disclosure relates to therapeutic combinations comprising an anti-KMA antibody and a proteasome inhibitor.

The phrase “anti-KMA antibody” is used in the context of the present disclosure to refer to an antibody that binds or specifically binds Kappa Myeloma Antigen. Kappa Myeloma Antigen (KMA) is a membrane-bound light chain with selectivity for kappa myeloma cells (Boux, H A. et al. (1983) J Exp Med. 158:1769).

In an example, an anti-KMA antibody is capable of binding KMA bearing cells. In another example, an anti-KMA antibody is capable of killing KMA bearing cells. For example, anti-KMA antibodies according to the present disclosure can bind and kill KMA bearing malignant plasma cells. In an example, anti-KMA antibodies according to the present disclosure do not bind intact immunoglobulin. Put another way, exemplary anti-KMA antibodies do not recognise kappa light chains that are in association with Ig heavy chain such as in an intact Ig molecules.

For example, the anti-KMA antibody can be the “K121 antibody” disclosed in (Hutchinson et al. 2011) or a variant, antigen binding fragment or humanised form thereof. An exemplary humanized form is referred to in the context of the present disclosure as “kappamab”, an antibody having a heavy chain variable region (V_{H}) comprising the amino acid sequence set forth in SEQ ID NO: 1 and a light chain variable region (VL) comprising the amino acid sequence set forth in SEQ ID NO: 2. Thus, in an example, the anti-KMA antibody is kappamab.

In another example, the anti-KMA antibody binds to or specifically binds to an epitope of KMA that is specifically bound by kappamab or that competes with kappamab for binding to KMA, wherein kappamab has a VH comprising the amino acid sequence set forth in SEQ ID NO: 1 and a VL comprising the amino acid sequence set forth in SEQ ID NO: 2.

Kappamab binds an epitope of KMA located in the switch region of kappa light chain (SEQ ID NO: 3). Amino acid substitution in the epitope can increase binding affinity of kappamab (Hutchinson et al. 2011). Accordingly, in an example, an anti-KMA antibody according to the present disclosure competes with an antibody that binds or specifically binds a region comprising an amino acid sequence as shown in SEQ ID NO: 3 with at least one, at least two or at least three amino acid substitutions. In another example, an anti-KMA antibody according to the present disclosure competes with an antibody that binds or specifically binds an epitope comprising an amino acid sequence as shown in SEQ ID NO: 4 with at least one, at least two or at least three amino acid substitutions. Exemplary substitutions include conservative amino acid substitutions such as those described below in Table 1. In an example, aspartic acid (Asp (D)) in SEQ ID NO: 4 is substituted with glutamic acid (Glu (E)) (SEQ ID NO: 5). Accordingly, in an example, an anti-KMA antibody according to the present disclosure competes with an antibody that binds or specifically binds an epitope comprising an amino acid sequence as shown in SEQ ID NO: 5.

TABLE 1
Exemplary substitutions.

| Original | Exemplary |
|---|---|
| Residue | Substitutions |
| Arg (R) | Lys (K) |
| Glu (E) | Asp (D) |
| Ile (I) | Leu (L); Val (V); Ala (A) |
| Leu (L) | Ile (I); Val (V); Met (M); Ala |
| | (A); Phe (F) |
| Lys (K) | Arg (R) |

In another example, an anti-KMA antibody according to the present disclosure competes with an antibody that binds or specifically binds an epitope comprising an amino acid sequence as shown in SEQ ID NO: 4. In another example, an anti-KMA antibody according to the present disclosure competes with an antibody that binds or specifically binds an epitope consisting of the amino acid sequence as shown in SEQ ID NO: 4. In another example, an anti-KMA antibody according to the present disclosure competes with an antibody that binds or specifically binds an epitope consisting of the amino acid sequence as shown in SEQ ID NO: 5.

Antibodies may be identified by their ability to compete for binding to KMA or a region or epitope thereof using various methods known in the art. For example, antibody binding to KMA on kappa human myeloma cell lines (κHMCL) such as KMS-11, KMS-26 and JJN3 can be assessed (Asvadi et al. 2015). In this procedure, an anti-KMA antibody such as kappamab is conjugated with biotin using established procedures (Hofmann K, et al. (1982) Biochemistry 21: 978-84). Antibodies are then evaluated by their capacity to compete with the binding of the biotinylated kappamab antibody to KMA on κHMCL cells. The binding of biotinylated kappamab to κHMCL cells may be assessed by the addition of fluorescein-labelled streptavidin which will bind to biotin on the labelled antibody. Fluorescence staining of cells is then quantified by flow cytometry, and the competitive effect of antibodies expressed as a percentage of the fluorescence levels obtained in the absence of the competitor.

In another example, the anti-KMA antibody has a VH comprising the CDRs as shown in SEQ ID NO: 6, SEQ ID NO: 7 and SEQ ID NO: 8 and a VL. In another example, the anti-KMA antibody has a VH and a VL comprising CDRs as shown in SEQ ID NO: 9, SEQ ID NO: 10 and SEQ ID NO: 11. In another example, the anti-KMA antibody has a VH comprising CDRs as shown in SEQ ID NO: 6, SEQ ID NO: 7 and SEQ ID NO: 8 and a VL comprising CDRs as shown in SEQ ID NO: 9, SEQ ID NO: 10 and SEQ ID NO: 11. In another example, the anti-KMA antibody has a VH comprising CDRs as shown in SEQ ID NO: 6, SEQ ID NO: 7 SEQ ID NO: 8, an amino acid sequence at least 90%, at least 95%, at least 98%, at least 99% identical to SEQ ID NO: 1 and a VL comprising CDRs as shown in SEQ ID NO: 9, SEQ ID NO: 10, SEQ ID NO: 11 and an amino acid sequence at least 90%, at least 95%, at least 98%, at least 99% identical to SEQ ID NO: 2.

In another example, the anti-KMA antibody has a VH comprising the amino acid sequence shown in SEQ ID NO: 1 and a VL comprising the amino acid sequence shown in SEQ ID NO: 2.

In another example, the anti-KMA antibody has the CDRs shown in SEQ ID NO: 1 and SEQ ID NO: 2, wherein the CDRs are assigned using the Kabat numbering system. In another example, the anti-KMA antibody has the CDRs shown in SEQ ID NO: 1 and SEQ ID NO: 2, wherein the CDRs are assigned using the IMGT numbering system. In another example, the anti-KMA antibody has the CDRs shown in SEQ ID NO: 1 and SEQ ID NO: 2, wherein the CDRs are assigned using EU numbering system of Kabat.

In an example, the anti-KMA antibody is a naked antibody. In other examples, the anti-KMA antibody is a full-length antibody, intact antibody or whole antibody. In an example, the anti-KMA antibody is monospecific.

In another example, the anti-KMA antibody is an antigen binding fragment comprising CDRs as shown in SEQ ID NO: 6, SEQ ID NO: 7, SEQ ID NO: 8, SEQ ID NO: 9, SEQ ID NO: 10 and SEQ ID NO: 11.

In another example, the anti-KMA antibody has a VH comprising the amino acid sequence shown in SEQ ID NO: 12 or a humanised variant thereof and a VL comprising the amino acid sequence shown in SEQ ID NO: 13 or a humanised variant thereof.

In another example, the anti-KMA antibody has the CDRs shown in SEQ ID NO: 12 and SEQ ID NO: 13 or humanised variants thereof, wherein the CDRs are assigned using the Kabat numbering system. In another example, the anti-KMA antibody has the CDRs shown in SEQ ID NO: 12 and SEQ ID NO: 13 or humanised variants thereof, wherein the CDRs are assigned using the IMGT numbering system. In another example, the anti-KMA antibody has the CDRs shown in SEQ ID NO: 12 and SEQ ID NO: 13 or humanised variants thereof, wherein the CDRs are assigned using EU numbering system of Kabat.

The term “proteasome inhibitor” is used in the context of the present disclosure to refer to molecules that inhibit the action of proteasomes (i.e. cellular breakdown of proteins).

Various proteasome inhibitors are known in the art (e.g. see review by Crawford et al. 2011). In one example, the proteasome inhibitor inhibits CT-L activity of the proteasome. For example, the proteasome inhibitor can be bortezomib or cafilzomib.

In another example, the proteasome inhibitor is selected from the group consisting of marizomib, oprozomib, epoxomicin, salinosporamide A, carfilzomib, ixazomib and bortezomib.

For example, the proteasome inhibitor can be marizomib. In another example, the proteasome inhibitor is oprozomib. In another example, the proteasome inhibitor is epoxomicin. In another example, the proteasome inhibitor is carfilzomib. In another example, the proteasome inhibitor is salinosporamide A. In another example, the proteasome inhibitor is delanzomib. In another example, the proteasome inhibitor is bortezomib. In another example, the proteasome inhibitor is ixazomib.

In an example, therapeutic combinations are provided for simultaneous administration. In another example, therapeutic combinations are provided for sequential administration. In another example, therapeutic combinations are provided as a composition. In these examples, the anti-KMA antibody, proteasome inhibitor or composition thereof can be formulated with a pharmaceutically acceptable carrier and/or excipient.

Therapeutic combinations according to the present disclosure are formulated to comprise a therapeutic level or dose of an anti-KMA antibody and a proteasome inhibitor.

In an example, the dose of the anti-KMA antibody ranges from about 0.1 mg/kg to about 90 mg/kg. In another example, the dose of the anti-KMA antibody ranges from about 0.2 mg/kg to about 60 mg/kg. In another example, the dose of the anti-KMA antibody ranges from about 0.3 mg/kg to about 30 mg/kg. In another example, the dose of the anti-KMA antibody ranges from about 1 mg/kg to about 20 mg/kg. In another example, the dose of the anti-KMA antibody ranges from about 3 mg/kg to about 10 mg/kg. In another example, the dose of the anti-KMA antibody is 3 mg/kg. In another example, the dose of the anti-KMA antibody is 10 mg/kg.

The appropriate dose of the proteasome inhibitor will depend on the proteasome inhibitor being administered. Exemplary general doses of proteasome inhibitors range from about 0.5 mg/m² to about 1.5 mg/m². In an example, the dose of the proteasome inhibitor ranges from about 0.7 mg/m² to about 1.3 mg/m². In another example, the dose of the proteasome inhibitor is about 0.7 mg/m². In another example, the dose of the proteasome inhibitor is about 1.0 mg/m². In another example, the dose of the proteasome inhibitor is about 1.3 mg/m².

Appropriate proteasome inhibitor dosing can also be obtained from prescribing information. For example, bortezomib can be administered at 1.3 mg/m², reduced to 1.0 mg/m² and 0.7 mg/m² if there are specific side effects.

Exemplary doses provided in therapeutic combinations according to the present disclosure are discussed further below.

In another example, the therapeutic combination further comprises one or more additional anti-cancer agents. Exemplary anti-cancer agents include chemotherapy, immunomodulatory drugs such as thalidomide, lenalidomide and pomalidomide, histone deacetylase inhibitors such as panobinostat or vorinostat, antibodies such as elotuzumab, daratumumab, isatuximab and anti-PD1 antibodies such as pembrolizumab, nivolumab and atezolizumab, or, a steroid such as dexamethasone.

In an example, the additional anti-cancer agent is dexamethasone. In another example, the additional anti-cancer agent is lenalidomide.

In an example, the therapeutic combination comprises at least two additional anti-cancer agents. For example, the additional anti-cancer agents can be dexamethasone and lenalidomide. In other examples, the therapeutic combination comprises at least three, at least four, at least five, at least six additional anti-cancer agents.

### Method of Treatment

In an example, the methods of the present disclosure relate to the treatment multiple myeloma and related pathologies, the methods comprising administering an anti-KMA antibody and a proteasome inhibitor. For example, the methods can comprise administering an above referenced therapeutic combination.

As used herein, the terms “treating”, “treat” or “treatment” include administering a therapeutically effective amount of an anti-KMA antibody and a proteasome inhibitor to reduce or delay the onset or progression of disease, or to reduce or eliminate at least one symptom of disease.

The terms “multiple myeloma” or “myeloma” are used in the context of the present disclosure to refer to cancer of plasma cells. In the context of the present disclosure, these terms encompasses secretory myeloma, non-secretory myeloma, light chain only myeloma, smouldering myeloma and related pathologies. Exemplary related pathologies include plasmacytoma, amyloidosis, monoclonal gammopathy of undetermined significance.

Subjects with multiple myeloma can be characterised into various subject populations. Exemplary populations are described in (Rajkumar et al. 2011).

In an example, a subjects multiple myeloma can be characterised as progressive disease (Rajkumar et al. 2011). Put another way, the methods of the present disclosure relate to the treatment of progressive multiple myeloma in a subject. Exemplary indicators of “progressive disease” include an increase of about 25% from the lowest response value in any one of the following: Serum M-component (absolute increase > or equal to 0.5 g/dL) and/or Urine M-component (absolute increase must be > or equal to 200 mg/24 hr. Other exemplary indicators include definite development of new bone lesions or soft tissue plasmacytomas or definite increase in the size of existing bone lesions or soft tissue plasmacytomas; development of hypercalcemia (corrected serum calcium>11.5 mg/dL) that can be attributed solely to the multiple myeloma. In an example, the subjects multiple myeloma has relapsed and is characterised as progressive disease. In this example, the subjects multiple myeloma can also be refractory to therapy.

In an example, the subjects multiple myeloma has relapsed. “Relapsed myeloma” is used to refer to previously treated myeloma that progresses and requires the initiation of salvage therapy but does not meet criteria for either “primary refractory myeloma”.

In another example, the subject has primary refractory myeloma. “Primary refractory myeloma” is used to refer to disease that is nonresponsive in patients who have never achieved a minimal response or better with any therapy.

In another example, the subject has refractory myeloma. The term “refractory myeloma” is used to refer to disease that is nonresponsive while on primary or salvage therapy, or progresses within 60 days of last therapy. In an example, a subjects multiple myeloma is refractory to an anti-cancer therapy. The term “refractory” is used in this context to refer to a line of anti-cancer therapy that is no longer therapeutically effective against a subject's multiple myeloma. For example, a subject treated by the methods of the present disclosure can be refractory to at least one proteasome inhibitor. For example, a subject can be refractory to bortezomib. A “line of therapy” is defined as one or more cycles of a planned treatment program. This may consist of one or more planned cycles of single-agent therapy or combination therapy, as well as a sequence of treatments administered in a planned manner. For example, a planned treatment approach of induction therapy followed by autologous stem cell transplantation, followed by maintenance is considered one line of therapy.

In another example, subjects are refractory to at least two prior lines of therapy. In another example, a subject may be refractory to at least three, at least four, at least five, at least six prior lines of therapy. In this example, at least one line of therapy may be bortezomib.

In another example, the subject has relapsed and refractory myeloma. “Relapsed and refractory myeloma” is used to refer to disease that is nonresponsive while on salvage therapy, or progresses within 60 days of last therapy in patients who have achieved minimal response (MR) or better at some point previously before then progressing in their disease course.

In an example, the multiple myeloma treated according to the present disclosure is characterised as stable disease at the time of first administration. Put another way, subjects can be in plateau phase at the time of first administration. Exemplary criteria for stable disease can include stabilization of the M-protein without further tumour regression despite continued treatment, few or no symptoms from myeloma and/or no blood transfusion requirement (Blade et al. 1998).

Subjects treated according to the methods of the present disclosure have multiple myeloma or a related pathology encompassed by the present disclosure.

In an example, a subject treated according to the present disclosure has received at least one line of prior therapy for their multiple myeloma. For example, a subjects multiple myeloma can have relapsed. In another example, a subject has received at least two, at least three, at least four, at least five, at least six prior lines of therapy. In these examples, a subject can have achieved at least a minimal response (about 25% reduction in M protein) to their most recent line of therapy.

In another example, a subject has serum kappa free light chain levels less than about 350 mg/ml. In another example, a subject has serum kappa free light chain levels less than about 300 mg/ml. In another example, a subject has serum kappa free light chain levels less than about 275 mg/ml. In another example, a subject has serum kappa free light chain levels less than about 250 mg/ml.

In another example, the methods of the present disclosure also relate to treating multiple myeloma in subjects with high serum cytokine levels. For example, the methods of the present disclosure relate to treating multiple myeloma in a subject, the method comprising selecting a subject who has high serum levels of one or more of the following factors relative to control serum levels: hepatocyte growth factor (HGF), macrophage inhibitory factor (MIF), CCL27, G-CSF, CXCL9, and CXCL10; and administering to the subject an anti-KMA antibody. In one aspect of the examples herein, serum analyte levels described herein are determined by immunoassay.

In an example, a high serum level of HGF is above about 0.5 ng/ml. In an example, a high serum level of HGF is above about 0.6 ng/ml, about 0.7 ng/ml, about 0.8 ng/ml, about 0.9 ng/ml, about 1.0 ng/ml, about 1.1 ng/ml, about 1.2 ng/ml, about 1.3 ng/ml, about 1.4 ng/ml, about 1.5 ng/ml. In another example, a high serum level of HGF is at least about 1.6 ng/ml.

In another example, a high serum level of MIF is above about 5000 pg/ml. In another example, a high serum level of MIF is above about 5200 pg/ml, about 5400 pg/ml, about 5600 pg/ml, about 5800 pg/ml, about 6000 pg/ml, about 6200 pg/ml, about 6400 pg/ml, about 6600 pg/ml, about 6800 pg/ml, about 7200 pg/ml.

In another example, a high serum level of CCL27 is above about 500 pg/ml. In another example, a high serum level of CCL27 is above about 600 pg/ml, about 700 pg/ml, about 800 pg/ml, about 900 pg/ml, about 1000 pg/ml, about 1100 pg/ml, about 1200 pg/ml, about 1300 pg/ml, about 1400 pg/ml, about 1500 pg/ml.

In another example, a high serum level of G-CSF is above about 55 pg/ml. In another example, a high serum level of G-CSF is above about 65 pg/ml, about 75 pg/ml, about 85 pg/ml, about 95 pg/ml, about 105 pg/ml, about 115 pg/ml, about 125 pg/ml, about 135 pg/ml, about 145 pg/ml, about 155 pg/ml.

In another example, a high serum level of CXCL9 is above about 550 pg/ml. In another example, a high serum level of CXCL9 is above about 600 pg/ml, about 650 pg/ml, about 700 pg/ml, about 750 pg/ml, about 800 pg/ml, about 850 pg/ml, about 900 pg/ml, about 950 pg/ml, about 1000 pg/ml, about 1050 pg/ml.

In another example, a high serum level of CXCL10 is above about 850 pg/ml. In another example, a high serum level of CXCL10 is above about 900 pg/ml, about 950 pg/ml, about 1000 pg/ml, about 1050 pg/ml, about 1100 pg/ml, about 1150 pg/ml, about 1200 pg/ml, about 1250 pg/ml, about 1300 pg/ml, about 1350 pg/ml.

High serum cytokine levels are determined in a sample obtained from the subject.

### Administration

In an example, the anti-KMA antibody and proteasome inhibitor are administered as a single composition.

In another example, the anti-KMA antibody and proteasome inhibitor are administered as separate compositions. For example, the anti-KMA antibody and proteasome inhibitor can be administered simultaneously. In another example, the anti-KMA antibody and proteasome inhibitor can be administered sequentially. In this example, administration of the anti-KMA antibody and proteasome inhibitor is carried out over a defined time period (usually minutes, hours or days). In an example, the period between sequential administration can be several days, provided that there is still sufficient levels of the first therapeutic to provide or add to the therapeutic benefit of the second therapeutic when it is administered. In one example, administration of an anti-KMA antibody is followed by sequential administration of a proteasome inhibitor. In another example, administration of a proteasome inhibitor is followed by sequential administration of an anti-KMA antibody.

Therapeutic combinations according to the present disclosure can be administered via various routes. Exemplary routes of administration include intravenous administration as a bolus or by continuous infusion over a period of time, intramuscular, intraperitoneal, intracerobrospinal, intrathecal, oral routes.

In an example, the anti-KMA antibody and proteasome inhibitor are administered via the same route. For example, both the anti-KMA antibody and proteasome inhibitor can be administered intravenously via continuous infusion. In another example, the anti-KMA antibody and proteasome inhibitor are administered via different routes. For example, the anti-KMA antibody can administered intravenously via continuous infusion and the proteasome inhibitor can be administered orally.

In an example, intravenous infusion of an anti-KMA antibody will last about one to two hours. In another example, a constant infusion of anti-KMA antibody may be provided to maintain a constant level of the antibody in serum.

In an example, anti-KMA antibody is administered weekly. In another example, anti-KMA antibody is administered monthly. In an example, anti-KMA antibody can be administered weekly and then monthly thereafter. For example, anti-KMA antibody can be administered weekly for at least four, at least five, at least six, at least seven, at least eight, at least nine, at least 10 weeks and then monthly thereafter. In an example, anti-KMA antibody can be administered weekly for about 8 weeks and then monthly thereafter. In these examples, weekly administration can be referred to as an induction dose and the monthly administration is referred to as a maintenance dose. The dose of anti-KMA antibody may be increased or decreased throughout the treatment regimen.

One of skill in the art would appreciate that proteasome inhibitors are generally administered in cycles. For example, Salinosporamide A and marizomib can be given as an intravenous infusion at about 0.5 mg/m² every 4 days of a 21 day cycle. In another example, opozomib can be given as an oral drug at 240 mg per day, once daily. In another example, Ixazomib can be given orally at about 4 mg weekly on days 1, 8, and 15 of a 28-day cycle.

Subject to tolerance by the subject, dose can be progressively increased or decreased over time. For example, carfilzomib can be given as an intravenous infusion at about 20 mg/m² in Cycle 1 on Days 1 and 2. If tolerated, the dose can be escalated to 27 mg/m² on Day 8 of Cycle 1. In another example, bortezomib is administered as three weekly cycles of two doses per week for the first two weeks followed by a treatment free week. In this example, the initial dose of the bortezomib can be about 1.3 mg/m² with dose reduction to 1.0 mg/m² and 0.7 mg/m² if higher doses are not tolerated.

In an example, dosing can be adjusted based on clinical evaluation or, if appropriate, prescribing information. Exemplary clinical evaluation may include physical examination, assessment of haematological toxicity (e.g. determine Grade 4 neutropenia or thrombocytopenia, or thrombocytopenia) and/or assessment of platelet count.

In another example, it may be desirable to obtain a sample from a subject post administration of an anti-KMA antibody to confirm that an analyte is present at a particular level in the sample before sequentially administering a proteasome inhibitor to the subject. Exemplary analytes include serum levels of hepatocyte growth factor (HGF), macrophage inhibitory factor (MIF), CCL27, G-CSF, CXCL9, and CXCL10.

In an example, the proteasome inhibitor is sequentially administered when serum levels of HGF, MIF, CCL27, G-CSF, CXCL9, or CXCL10 in the subject are equivalent to serum levels in a healthy adult. Exemplary serum levels in healthy adults are shown in Table 2.

TABLE 2
Adult serum cytokine levels (median and ranges)
for healthy adults and myeloma patients.

| | Median serum levels in | |
|---|---|---|
| | healthy adults | |
| Cytokine | (Range) | |
| CXCL9 (MIG) | ¹106.2 pg/mL | (51-390.6) |
| | ²278.2 pg/mL | (138.7-539.9) |
| CXCL10 (IP10) | ²576.2 pg/mL | (368.5-808.5) |
| HGF | ³0.44 ng/mL | (0.18-0.69) |
| | 319.7 pg/mL ² | (196.6-477.9) |
| MIF | ⁴(414 pg/ml-4707 pg/ml) | |
| CCL27 (CTACK) | ²335.9 pg/mL | (190.9-468.6) |
| G-CSF | ²45.5 pg/mL | (34-53.6) |

In an example, the proteasome inhibitor is sequentially administered when serum levels of HGF are between about 0.10 ng/ml and 0.90 ng/ml. In an example, the proteasome inhibitor is sequentially administered when serum levels of HGF are between about 0.15 ng/ml and 0.80 ng/ml. In an example, the proteasome inhibitor is sequentially administered when serum levels of HGF are between about 0.18 ng/ml and 0.69 ng/ml.

In an example, the proteasome inhibitor is sequentially administered when serum levels of CCL27 are between about 120 pg/ml and 650 pg/ml. In an example, the proteasome inhibitor is sequentially administered when serum levels of CCL27 are between about 150 pg/ml and 550 pg/ml. In an example, the proteasome inhibitor is sequentially administered when serum levels of CCL27 are between about 190 pg/ml and 470 pg/ml.

In an example, the proteasome inhibitor is sequentially administered when serum levels of G-CSF are between about 20 pg/ml and 100 pg/ml. In an example, the proteasome inhibitor is sequentially administered when serum levels of G-CSF are between about 25 pg/ml and 80 pg/ml. In an example, the proteasome inhibitor is sequentially administered when serum levels of G-CSF are between about 30 pg/ml and 55 pg/ml.

In an example, the proteasome inhibitor is sequentially administered when serum levels of CXCL9 are between about 40 pg/ml and 700 pg/ml. In an example, the proteasome inhibitor is sequentially administered when serum levels of CXCL9 are between about 45 pg/ml and 600 pg/ml. In an example, the proteasome inhibitor is sequentially administered when serum levels of CXCL9 are between about 50 pg/ml and 550 pg/ml.

In an example, the proteasome inhibitor is sequentially administered when serum levels of CXCL10 are between about 200 pg/ml and 1.1 ng/ml. In an example, the proteasome inhibitor is sequentially administered when serum levels of CXCL10 are between about 250 pg/ml and 900 pg/ml. In an example, the proteasome inhibitor is sequentially administered when serum levels of CXCL10 are between about 300 pg/ml and 850 pg/ml.

In an example, the proteasome inhibitor is sequentially administered when serum levels of MIF are between about 350 pg/ml and 5000 pg/ml. In an example, the proteasome inhibitor is sequentially administered when serum levels of MIF are between about 400 pg/ml and 4000 pg/ml. In an example, the proteasome inhibitor is sequentially administered when serum levels of HGF are between about 420 pg/ml and 2000 pg/ml.

### Additional Steps

As will be appreciated by those skilled in the art, some myeloma patients have significant levels of free kappa light chain in their circulation. Accordingly, in one example, methods of treatment further comprise treating a subject to reduce the levels of free kappa light chains circulating in their fluid (e.g. blood) prior to administration of an anti-KMA antibody. This additional treatment step may involve, for example, plasmapherisis. As will be known by those skilled in the art, plasmapherisis is a process in which the plasma is removed from blood cells by a device known as a cell separator. The separator works either by spinning the blood at high speed to separate the cells from the fluid or by passing the blood through a membrane with pores so small that only the plasma can pass through. The cells are returned to the subject, while the plasma, which contains the free kappa light chains, is discarded and replaced with other fluids. Medication to keep the blood from clotting (e.g. an anticoagulant) may be given through a vein during the procedure.

In another example, the methods of the present disclosure further comprise administering one or more additional anti-cancer agents. Exemplary anti-cancer agents include chemotherapy, immunomodulatory drugs such as thalidomide, lenalidomide and pomalidomide, histone deacetylase inhibitors such as panobinostat or vorinostat, antibodies such as elotuzumab, daratumumab, isatuximab and anti-PD1 antibodies such as pembrolizumab, nivolumab and atezolizumab, or, a steroid such as dexamethasone. In an example, the additional anti-cancer agent is dexamethasone. In another example, the additional anti-cancer agent is lenalidomide.

In an example, at least two additional anti-cancer agents are administered. For example, dexamethasone and lenalidomide can be administered. In other examples, at least three, at least four, at least five, at least six additional anti-cancer agents are administered.

### Treating Subjects Refractory to Therapy

Subject's refractory to therapy can have high serum levels of analytes such as HGF, MIF, CCL27, G-CSF, CXCL9, and CXCL10. The present inventors have identified that administration of kappamab can reduce serum levels of these analytes towards serum levels observed in healthy subjects. Accordingly, in an example, the methods of the present disclosure encompass treating subjects that have high serum levels of HGF, MIF, CCL27, G-CSF, CXCL9 or CXCL10 relative to control serum levels by administering an anti-KMA antibody.

The term “high levels” is used to refer to serum levels above those observed in healthy subjects. Exemplary serum levels in healthy subjects are shown above in Table 2.

In an example, subjects treated with methods according to the present disclosure can have high serum levels of HGF. In an example, subjects can have serum levels of HGF above about 0.3 ng/ml. In an example, subjects can have serum levels of HGF above about 0.4 ng/ml. In another example, subjects can have serum levels of HGF above about 0.5 ng/ml. In another example, subjects can have serum levels of HGF above about 0.6 ng/ml. In another example, subjects can have serum levels of HGF above about 0.7 ng/ml. In another example, subjects can have serum levels of HGF above about 0.8 ng/ml. In another example, subjects can have serum levels of HGF above about 0.9 ng/ml. In another example, subjects can have serum levels of HGF of about 1 ng/ml. In another example, subjects can have serum levels of HGF of above about 1 ng/ml. In another example, subjects can have serum levels of HGF of about 1.1 ng/ml. In another example, subjects can have serum levels of HGF of about 1.2 ng/ml. In another example, subjects can have serum levels of HGF of about 1.3 ng/ml. In another example, subjects can have serum levels of HGF of about 1.4 ng/ml. In another example, subjects can have serum levels of HGF of about 1.5 ng/ml. In another example, subjects can have serum levels of HGF of about 1.6 ng/ml.

In another example, subjects can have serum levels of HGF ranging from about 0.3 ng/ml to about 1.6 ng/ml. In another example, subjects can have serum levels of HGF ranging from about 0.4 ng/ml to about 1.0 ng/ml. In another example, subjects can have serum levels of HGF ranging from about 0.4 ng/ml to about 0.8 ng/ml.

In another example, subjects treated with methods according to the present disclosure can have high serum levels of CCL27. In another example, subjects can have serum levels of CCL27 above about 150 pg/ml. In another example, subjects can have serum levels of CCL27 above about 200 pg/ml. In another example, subjects can have serum levels of CCL27 above about 250 pg/ml. In another example, subjects can have serum levels of CCL27 above about 300 pg/ml. In another example, subjects can have serum levels of CCL27 above about 350 pg/ml. In another example, subjects can have serum levels of CCL27 above about 400 pg/ml. In another example, subjects can have serum levels of CCL27 above about 450 pg/ml. In another example, subjects can have serum levels of CCL27 above about 500 pg/ml. In another example, subjects can have serum levels of CCL27 above about 550 pg/ml. In another example, subjects can have serum levels of CCL27 above about 600 pg/ml.

In another example, subjects can have serum levels of CCL27 ranging from about 150 pg/ml to about 600 pg/ml. In another example, subjects can have serum levels of CCL27 ranging from about 170 pg/ml to about 550 pg/ml. In another example, subjects can have serum levels of CCL27 ranging from about 180 pg/ml to about 500 pg/ml.

In another example, subjects treated with methods according to the present disclosure can have high serum levels of G-CSF. In another example, subjects can have serum levels of G-CSF above about 20 pg/ml. In another example, subjects can have serum levels of G-CSF above about 25 pg/ml. In another example, subjects can have serum levels of G-CSF above about 30 pg/ml. In another example, subjects can have serum levels of G-CSF above about 35 pg/ml. In another example, subjects can have serum levels of G-CSF above about 40 pg/ml. In another example, subjects can have serum levels of G-CSF above about 45 pg/ml. In another example, subjects can have serum levels of G-CSF above about 50 pg/ml. In another example, subjects can have serum levels of G-CSF above about 55 pg/ml. In another example, subjects can have serum levels of G-CSF above about 65 pg/ml. In another example, subjects can have serum levels of G-CSF above about 65 pg/ml.

In another example, subjects can have serum levels of G-CSF ranging from about 20 pg/ml to about 65 pg/ml. In another example, subjects can have serum levels of G-CSF ranging from about 25 pg/ml to about 50 pg/ml. In another example, subjects can have serum levels of G-CSF ranging from about 30 pg/ml to about 55 pg/ml.

In another example, subjects treated with methods according to the present disclosure can have high serum levels of CXCL9. In another example, subjects can have serum levels of CXCL9 above about 70 pg/ml. In another example, subjects can have serum levels of CXCL9 above about 110 pg/ml. In another example, subjects can have serum levels of CXCL9 above about 150 pg/ml. In another example, subjects can have serum levels of CXCL9 above about 190 pg/ml. In another example, subjects can have serum levels of CXCL9 above about 230 pg/ml. In another example, subjects can have serum levels of CXCL9 above about 270 pg/ml. In another example, subjects can have serum levels of CXCL9 above about 310 pg/ml. In another example, subjects can have serum levels of CXCL9 above about 350 pg/ml. In another example, subjects can have serum levels of CXCL9 above about 390 pg/ml. In another example, subjects can have serum levels of CXCL9 above about 430 pg/ml. In another example, subjects can have serum levels of CXCL9 above about 470 pg/ml. In another example, subjects can have serum levels of CXCL9 above about 510 pg/ml. In another example, subjects can have serum levels of CXCL9 above about 550 pg/ml.

In another example, subjects can have serum levels of CXCL9 ranging from about 70 pg/ml to about 550 pg/ml. In another example, subjects can have serum levels of CXCL9 ranging from about 100 pg/ml to about 550 pg/ml. In another example, subjects can have serum levels of CXCL9 ranging from about 130 pg/ml to about 540 pg/ml.

In another example, subjects treated with methods according to the present disclosure can have high serum levels of CXCL10. In another example, subjects can have serum levels of CXCL10 above about 300 pg/ml. In another example, subjects can have serum levels of CXCL10 above about 350 pg/ml. In another example, subjects can have serum levels of CXCL10 above about 400 pg/ml. In another example, subjects can have serum levels of CXCL10 above about 450 pg/ml. In another example, subjects can have serum levels of CXCL10 above about 500 pg/ml. In another example, subjects can have serum levels of CXCL10 above about 550 pg/ml. In another example, subjects can have serum levels of CXCL10 above about 600 pg/ml. In another example, subjects can have serum levels of CXCL10 above about 650 pg/ml. In another example, subjects can have serum levels of CXCL10 above about 700 pg/ml. In another example, subjects can have serum levels of CXCL10 above about 750 pg/ml. In another example, subjects can have serum levels of CXCL10 above about 800 pg/ml. In another example, subjects can have serum levels of CXCL10 above about 850 pg/ml. In another example, subjects can have serum levels of CXCL10 above about 900 pg/ml.

In another example, subjects can have serum levels of CXCL10 ranging from about 300 pg/ml to about 900 pg/ml. In another example, subjects can have serum levels of CXCL10 ranging from about 320 pg/ml to about 900 pg/ml. In another example, subjects can have serum levels of CXCL10 ranging from about 350 pg/ml to about 850 pg/ml.

In another example, subjects treated with methods according to the present disclosure can have high serum levels of MIF. In another example, subjects can have serum levels of MIF above about 4.7 ng/ml. In another example, subjects can have serum levels of MIF above about 4.75 ng/ml. In another example, subjects can have serum levels of MIF above about 5 ng/ml. In another example, subjects can have serum levels of MIF above about 5.5 ng/ml. In another example, subjects can have serum levels of MIF above about 6 ng/ml.

In an example, the above exemplified methods further comprise administering a proteasome inhibitor such as bortezomib.

### EXAMPLES

### Example 1—Materials and Methods

### Patients

Patients 18 years; with kappa-restricted multiple myeloma who had an Eastern Cooperative Oncology Group (ECOG) performance status <2, had received at least 3 prior lines of therapy, achieved at least a minimal response (>25% reduction in M protein) to their most recent treatment, and had demonstrated persisting stable disease for at least 3 months were eligible for the study. Patients on maintenance therapy (thalidomide or lenalidomide) were also eligible for inclusion. Patients were excluded from the study if their serum κFLC was greater than 250 mg/L.

### Study Design

A 3+3 design was used to investigate the safety, dose limiting toxicity (DLT) and pharmacokinetics (PK) of kappamab, and to monitor for the formation of human anti-chimeric antibody (HACA) against kappamab. The pharmacodynamics of serum κFLC was also assessed. Drug-target modulation of cell signalling pathways was determined by measuring pro-inflammatory and anti-inflammatory cytokines, chemokines, and growth factors in patient serum at specific time intervals.

### Treatment Protocol

The study consisted of screening, treatment (Days 1-45), and follow-up (Days 46-135) phases. After providing HREC-approved consent, patients were screened and assessed for study eligibility. Up to a maximum of 30 patients were planned to be recruited in 5 kappamab dose cohorts (0.3 mg/kg, 1.0 mg/kg, 3.0 mg/kg, 10 mg/kg and 30 mg/kg) of 3 to 6 patients per cohort, depending on the occurrence of DLTs.

Within each dose cohort only 1 patient per week was to be treated. Provided that no patient experienced any DLT (defined as a >grade 3 non-haematological toxicity or grade 4 haematological toxicity using National Cancer Institute Common Toxicity Criteria Version 3.0) within 2 weeks of study drug administration, the first patient of the subsequent group of 3 patients commenced treatment at the next planned dose level of kappamab. All kappamab doses were administered as an intravenous (IV) infusion over 90 minutes. Premedication was not mandated, however, paracetamol (acetaminophen), corticosteroids, and antihistamines were allowed for the treatment of infusion reactions. All patients were followed up on Days 75, 105, and 135 post infusion for safety assessments.

### Safety

Evaluation of safety and tolerability during treatment included vital signs, physical examination, electrocardiogram (ECG), haematology assessments, clinical chemistry, C-reactive protein, β₂-microglobulin, immunoglobulin quantification, urinalysis, creatinine clearance, collection of adverse events (AEs), and drug-induced toxicity. All AEs were graded using the Cancer Therapy Evaluation Program Common Terminology Criteria for Adverse Events, version 3.0.

### Human Anti-Chimeric Antibody

Blood samples were taken at pre-infusion and at completion of the treatment phase (Day 45 post infusion) for measurement of HACA responses. The presence of HACA was measured using a validated sandwich ELISA. Briefly, kappamab was immobilized on ELISA plates and the captured antibodies were detected using biotinylated kappamab and ExtrAvidie-alkaline phosphatase (Sigma, USA) followed by the alkaline phosphatase (AP) substrate. Goat anti-human IgG was used as the positive control for the assay.

### Pharmacokinetics Analyses

The PK analysis was based on measurement of serum kappamab using an ELISA that was validated according to recommended guidelines. Briefly, immobilized human kappa Bence Jones protein (Bethyl, Sigma, USA) was used to capture kappamab. The captured kappamab was then detected using an AP-conjugated anti-human IgG (gamma chain specific; Sigma, Cat. No. H4522) followed by colour development with the AP substrate.

### Pharmacodynamic Analyses

Serum FLC levels were quantified using the FreeLite™ assay (The Binding Site Group Ltd, Birmingham, UK). Laboratory evaluation of patients' serum FLC levels in the presence of kappamab showed that the antibody does not interfere with nephelometry measurements (Table 3). Twenty-four-hour urine collection for urine protein electrophoresis immunofixation, urine FLC measurement (FreeLite™ assay), and creatinine clearance were collected on the day of infusion and on Day 45.

### Biomarker Analysis

Patient serum samples for biomarker analysis were collected approximately 30 minutes before infusion of kappamab and then at 6, 48, 192, and 1,080 hours after infusion for all patients. Samples were analysed in triplicate and serum levels of 48 human cytokines, chemokines, and growth factors were measured using the Bio-Plex Pro™ Human Cytokine 27-plex and 21-plex assays according to the manufacturer's recommendations (Bio-Rad, Carlsbad, Calif., USA; Cat No M10-0KCAF0Y, mg0-005KMII). Results were verified by repeating the 21-plex assay in triplicate and the samples were randomized across the assay plate. Pre-infusion samples were used as the baseline (t=0) for the analysis.

### Statistical Analysis

Multivariate analysis of the biomarker data was performed using R version 3.1.0 (2014-04-10) software (http://www.R-project.org/). Linear mixed effects modelling was completed using 1me4 (Bates et al. 2015). The significance of interactions terms, and interaction means were obtained using the Phia package (http://CRAN.R-project.org/package=phia). Multiple test correction was performed using the false discovery rate correction (Benjamini et al. 1995).

To find significance between kappamab dose, time, and cytokine expression levels, we fitted a linear mixed-effects model that modelled cytokine fluorescence expression levels with fixed-effects terms for baseline cytokine fluorescence expression, kappamab dose (with 4 levels: 0.3, 1.0, 3.0, and 10 mg/kg), time (treated as factor with 5 levels), and cytokine with 48 levels, plus their interactions, with a random effect associated with patient (12 levels) and plex (2 levels). Plex was used to distinguish between the two cytokine panels/plates. In R notation the statistical model is given as:

Val.y˜Val.x+Time.y*Dose*Protein+(1|Plex:Patient)

Here, Val.y represents cytokine expression at time points defined by time.y (ie, Time.y=6, 48, 192, and 1,080 hours after infusion). Val.x represents the baseline expression for each cytokine.

### Example 2—Clinical Assessment

### Kappamab Dose Cohorts

After review of the data up to Day 45 for patients in Cohorts 1 to 3 (0.1 mg/kg, 1.0 mg/kg, and 3.0 mg/kg dose cohorts), it was established that kappamab could be detected at all dose levels (up to Day 30).

### Patient Demographic and Baseline Data

A total of 12 patients (7 male, 5 female) with a median age of 63 years (range 47 to 83) were treated in the study (Table 4). Patients had received a median of 6 lines of prior antineoplastic therapy (range, 2 to 11); in addition 8/12 patients had received prior autologous stem cell transplant (ASCT). During the study, 9/12 patients received ongoing maintenance therapy with thalidomide, lenalidomide, and/or corticosteroids (Table 4).

### Safety

Eight of the 12 patients (67%) experienced treatment-emergent AEs and a total of 18 events were recorded and the most frequently reported AE was nausea (2/12 patients; 17%) (Table 5). The majority of AEs (17/18; 94%) were grade 1 or 2 and there was 1 grade 3 event (arthralgia; not related to study drug). Few patients 2/12 (17%) experienced study drug-related AEs (6 events) and these were at the highest dose level (10 mg/kg). One patient experienced grade 1 infusion reaction with flushing, dyspnea, and nausea after the start of infusion. The infusion was stopped and within 30 minutes the symptoms had resolved without treatment. The infusion was re-started and no further AEs were reported during the infusion. A second patient experienced grade 1 AEs on Day 2 post infusion with nausea, pain in extremity, and eructation (Table 5). The nausea resolved by Day 15, pain in extremity resolved by Day 8, and the eructation ceased by Day 15. There were no patients who experienced a DLT or discontinued the study due to an AE. Further, there were no deaths or other serious AEs reported during the study.

There were no dose-related trends in serum immunoglobulin levels, clinical chemistry, haematology, or urinalysis for patients during the study. On the basis of serum blood urea nitrogen, serum creatinine, and creatinine clearance there were no drug-related effects on kidney function. No changes from baseline were observed for lactate dehydrogenase levels or vital signs. The ECG data showed no prolongation of QTc intervals and there was no effect of kappamab on ECOG performance status scores. There was no apparent dose-related trend in serum C-reactive protein levels. No maximum tolerated dose of kappamab was defined in patients treated up to 10 mg/kg. Importantly, there was no evidence of immune complex formation or serum sickness in any patient treated with kappamab. Testing for HACA at Day 45 post infusion revealed that no antibody responses to kappamab were detected in any patient.

### Pharmacokinetic Evaluations

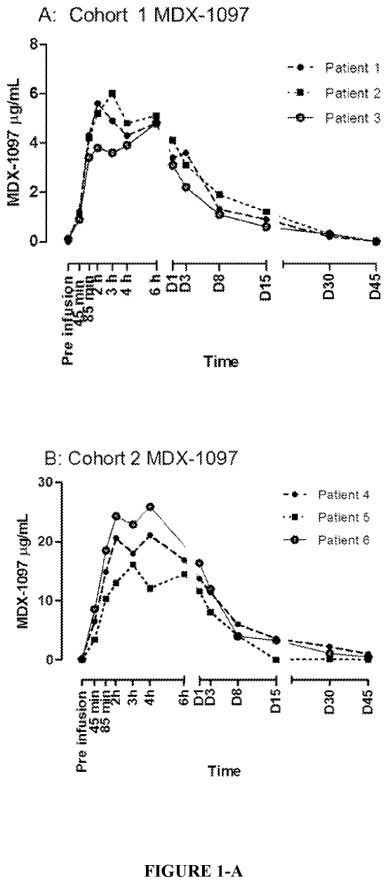

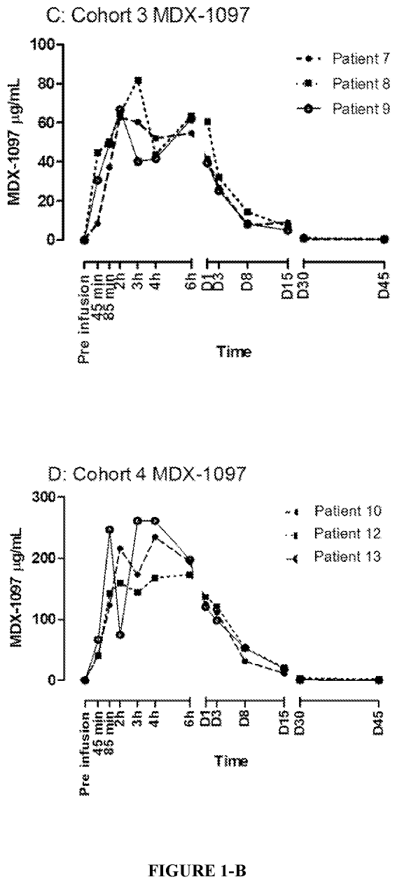

The PK parameters for kappamab are summarized in Table 6. Maximum serum concentrations of kappamab were achieved between 2 and 4 hours after the start of the IV infusion across all doses. After reaching maximal serum concentrations (Cₘₐₓ), kappamab appeared to decline in a biphasic manner, with the start of the apparent terminal elimination phase generally occurring 7 days after the start of the IV infusion. The mean apparent elimination half-life (t_{1/2}) became shorter with ascending dose, decreasing from 237 hours at 0.3 mg/kg to 124 hours at 10 mg/kg kappamab. Serum concentrations of kappamab were quantifiable in all patients until Day 30 post infusion (FIG. 1), across all of the doses (range: 0.3% to 10% of Cₘₐₓ at Day 30). The kappamab volume of distribution (V_{z}) was low and similar across ascending doses and is consistent with confinement of the antibody to the blood and extracellular fluid spaces (Table 6).

### Serum Kappa Free Light Chain Levels

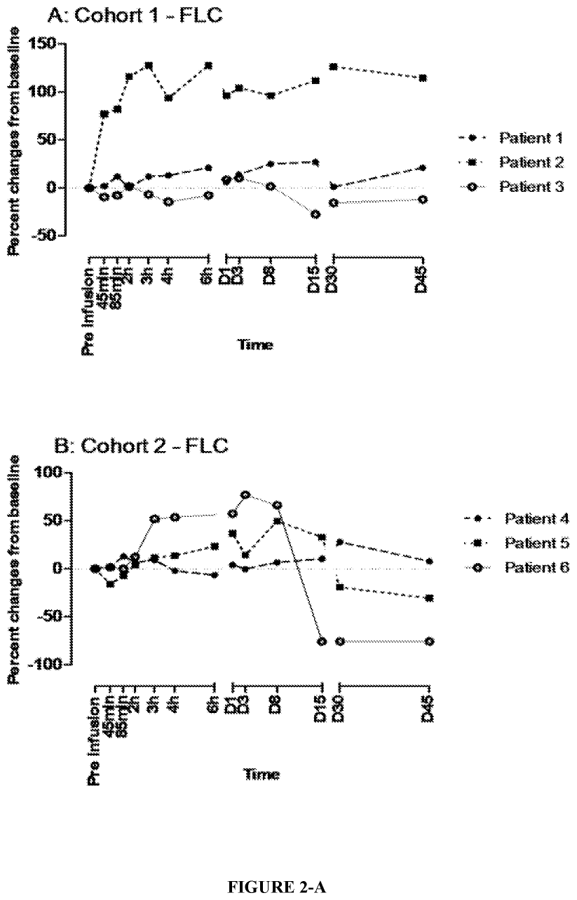

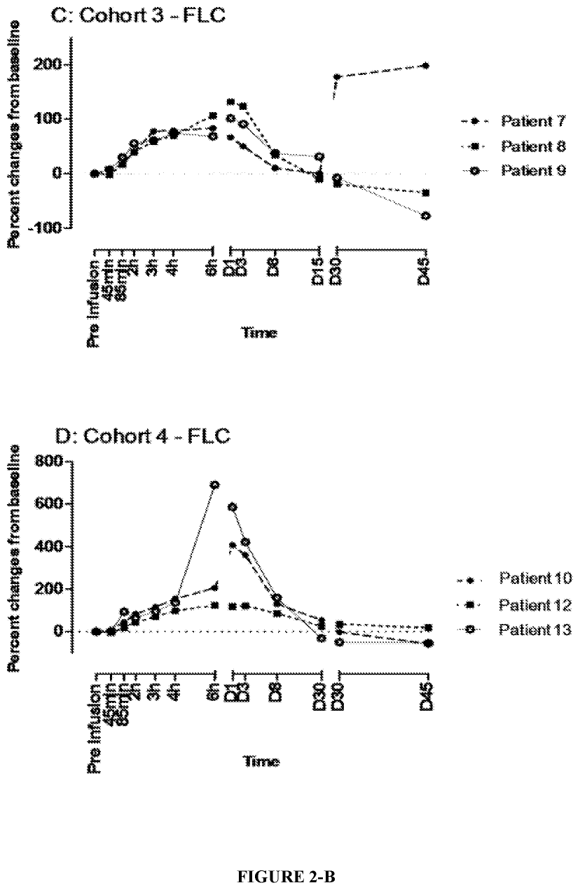

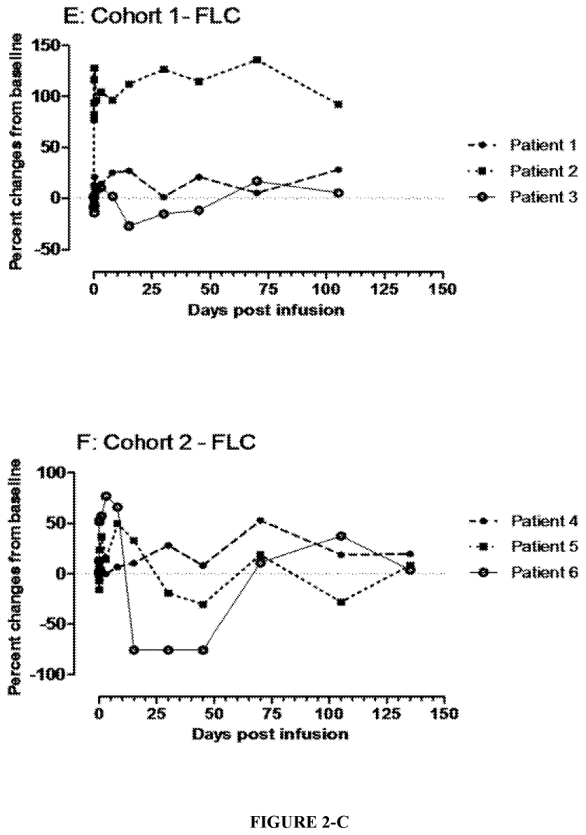

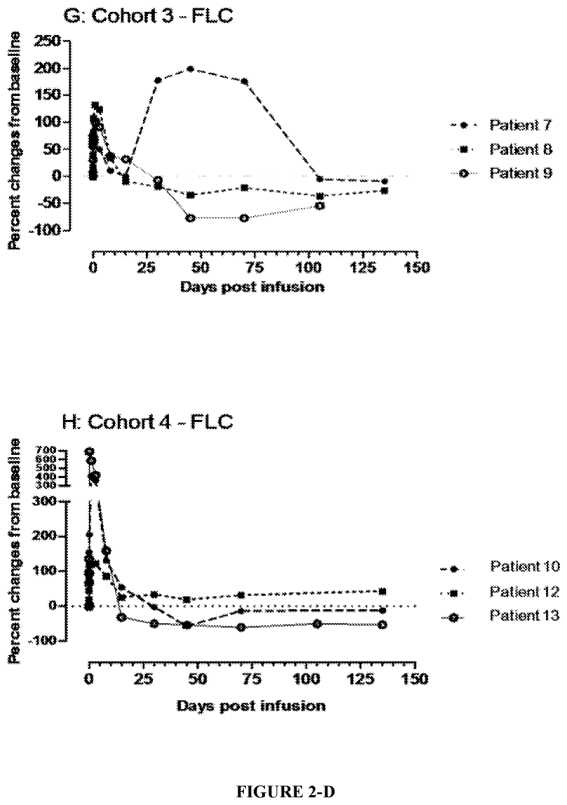

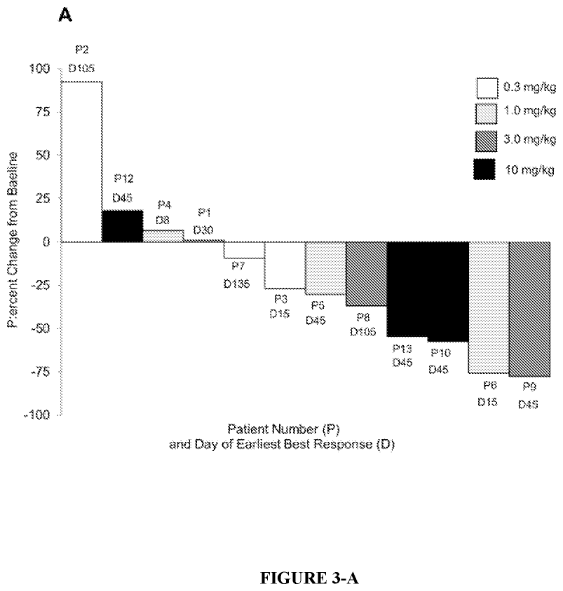

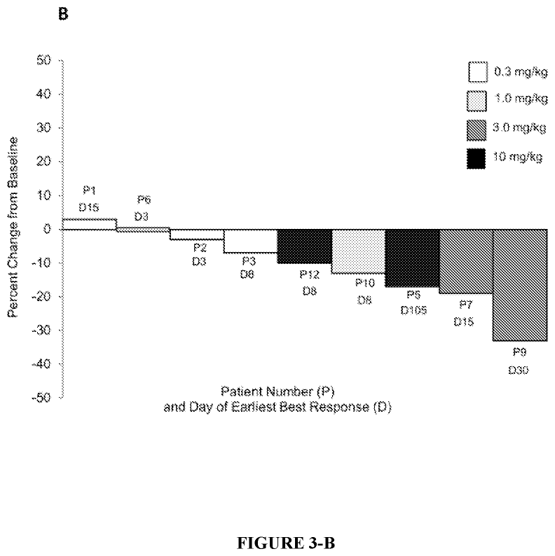

An increase in serum κFLC levels following the IV infusion of kappamab was observed in all patients with peak levels achieved between Days 1 and 15 (FIG. 2). Decreases in serum κFLC below baseline values were observed after Day 15 in the majority of patients (8/12; 67%) (FIG. 3-A), however, the greater magnitude of the decreases observed in some patients (Patients 6, 9, and 10) may be related to the low baseline κFLC levels in these patients (Table 7). Due to the small cohort size and the variability in baseline serum κFLC levels there was no clear dose dependency between the serum κFLC increase and kappamab concentration; however, the greatest increases were generally observed in the 2 highest dose groups (3.0 and 10 mg/kg).

Serum κFLC concentrations decreased between Days 8 and 30 and were generally similar to baseline values by Day 45 in the majority of patients, with the exception of Patients 2, 7, and 13 (FIG. 2). Patient 2 serum κFLC values were up to 136% higher than the baseline values during the study. Patient 7 serum κFLC values were 83% higher than baseline on Day 1 and then returned to the pre-infusion values on Day 15, followed by a 178% and 198% increase from baseline on Days 30 and 45, respectively. However, by Day 105 post infusion, the patient's κFLC values had returned to below baseline. Patient 13 serum κFLC values increased by 690% at 6 hours after IV infusion and then decreased steadily to 55% below baseline values on Day 45 and remained at this level for 3 months after Day 45 (FIG. 2; FIG. 3). There was no apparent change in serum λFLC concentration throughout the study and the changes in serum FLC ratios (κ:λ) were consistent with the fluctuations in serum κFLC concentrations.

### Urine Kappa Free Light Chain and Serum M Protein

There was large variability in urinary excretion of κFLC between patients at screening and on Day 1 and 45 post infusion (Table 7). On Day 1, 67% (8/12) of patients had a decrease in urine κFLC (range: 9% to 69% below baseline) and on Day 45 post infusion 58% (7/12) of patients had a decrease in κFLC (range 2% to 80% below baseline). Within each dose cohort there was no consistent dose-related trend in urinary excretion of κFLC throughout the study. During the study there were minor kappamab dose-related decreases in serum M protein levels in some patients (7/11 patients; 64%), however, these responses did not meet the IMWG response criteria (FIG. 3-B). Further, the magnitude of some of the decreases in M protein after treatment may have been associated with lower baseline levels of M protein in some patients (Table 7).

### Biomarker Analysis

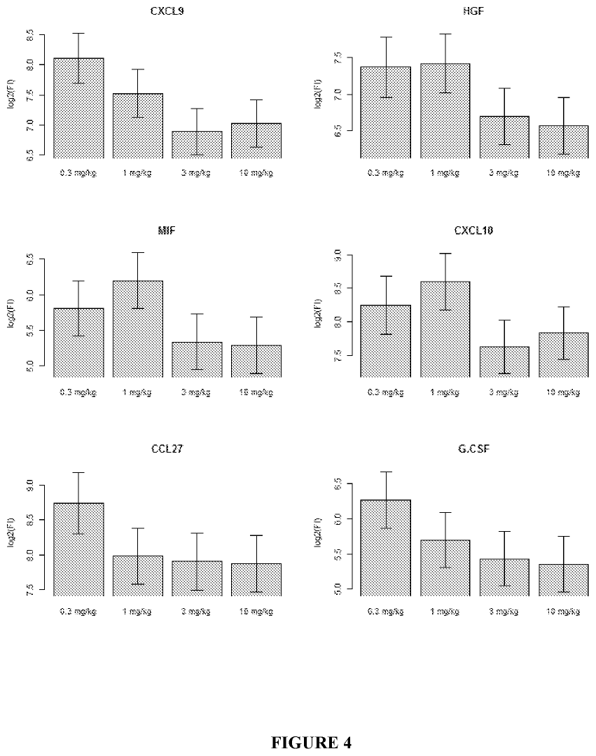

Results of the multivariate analysis of 48 different cytokines in patient serum over time and dose detected significant effects with the interactions kappamab dose:cytokine expression (P<0.001) but not with respect to the interactions between time:dose and time:cytokine see Table 8. Therefore, the interactions between kappamab dose and cytokine were explored and, after multiple test correction, 6 of the cytokines assessed showed statistically significant changes in expression with respect to kappamab dose (Table 9). After adjusting for cytokine baseline differences, kappamab dose-related decreases in serum concentration of the chemokines CXCL9 and CXCL10, macrophage inhibitory factor (MIF), HGF, CCL27, and granulocyte-colony stimulating factor (G-CSF) were observed (FIG. 4). Interestingly, the observed levels of CXCL10 contrast with the drug-induced increases observed in an elotuzumab phase I study (Zonder et al. (2012) Blood., 120, 552-9.

CXCL9, CXCL10, MIF, HGF, CCL27 and G-CSF play a significant role in the signalling pathways associated with survival of myeloma cells in the bone marrow microenvironment (BME). Increased serum levels of HGF, which is the cognate ligand for MET, an oncogenic tyrosine kinase, are a result of autocrine and paracrine pathways in the myeloma BME (Mahtouk et al. (2010) Biochim Biophys Acta., 1806, 208-19). The dysregulation of the HGF/MET signalling pathway in myeloma correlates with aggressive disease, drug resistance, and increased lytic bone lesions (Kristensen et al. (2013) Br J Haematol., 161, 373-82; Rocci et al. (2014) Br J Haematol, 164, 841-50). The inflammatory chemokines CXCL9 and CXCL10 are increased in multiple myeloma patient serum (Bolomsky (2016) Leuk Lymphoma., 1-10). Both chemokines are ligands for the CXCR3 receptor and depending on the receptor isoform (CXCR3A or CXCR3B), they up- or downregulate cell trafficking and proliferation (Muehlinghaus et al. (2005) Blood., 105, 3965-71). Unusually, myeloma cells express both receptor splice variants, but at different ratios and in a cell cycle-dependent manner (Giuliani et al. (2006) Haematologica., 91, 1489-97). Recently, the proinflammatory and atypical chemokine MIF was shown to bind CXCR4, resulting in recruitment of numerous cells including T cells, B cells, mesenchymal stromal cells, and cancer cells (Klasen et al. (2014) J Immunol., 192, 5273-84). The chemokine CCL27, which binds to the receptor CCR10, was also shown to induce chemotaxis of both normal and myeloma PCs (Nakayama et al. (2003) J Immunol, 170, 1136-40). While the cytokines described here have a broad range of immune effects, they all play a role in B cell trafficking and potential homing from secondary lymphoid organs to the BME.

No increases in serum cytokine concentrations were detected in response to kappamab treatment.

### Myeloma Response

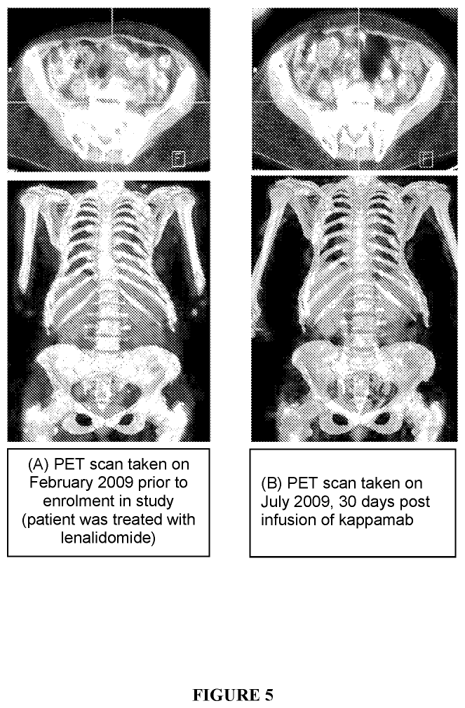

As patients only received a single infusion of kappamab it was not anticipated that any conventional responses would be observed. However, a ¹⁸fluorine-D-glucose (FDG)-positron emission tomography (¹⁸FDG-PET) scan following treatment in one patient with oligo-secretory light chain only multiple myeloma (Patient 8; 3 mg/kg kappamab dose group) showed an almost complete metabolic response when compared with a baseline study. Before treatment with kappamab the patient had extensive skeletal myelomatous disease (FIG. 5-A) but 30 days after kappamab treatment repeat scanning demonstrated significant resolution of previously noted FDG-avid lesions apart from an area of disease in the left femur, which exhibited reduced but incomplete resolution (FIG. 5-B). In addition, this patient showed symptomatic improvement including diminished bone pain and normalization of kidney function.

TABLE 3
Lack of interference of kappamab in the FreeLite ™ assay for measurement
of kappa and gamma free light chain. Comparison of kappa and gamma free
light chain measurements in patient pre-infusion samples spiked with increasing
concentrations of kappamab and control patient pre-infusion samples.

| | | FLC concentration measured in FreeLite ™ assay | | | |
|---|---|---|---|---|---|
| | | κFLC (mg/L) | | λFLC (mg/L) | |
| | Kappamab | Kappamab-spiked | Control | Kappamab-spiked | Control |
| Patient | added | pre-infusion | pre-infusion | pre-infusion | pre-infusion |
| number | (μg/mL) | sample | sample | sample | sample |
| 1 | 6 | 119 | 100 | 2.1 | 4.8 |
| | 0.0 | 113 | | 2.1 | |
| 2 | 6 | 352.5 | 191 | 2.1 | 4.7 |
| | 0.0 | 337.5 | | 2.1 | |
| 3 | 6 | 124 | 118 | 4.4 | 12.7 |
| | 0.0 | 122 | | 4.4 | |
| 4 | 20 | 29.1 | 33.1 | 2.1 | 4.3 |
| | 0.0 | 33.1 | | 2.1 | |
| 5 | 20 | 8.7 | 12.5 | 2.1 | NA |
| | 0.0 | 8.9 | | 2.1 | |
| 6 | 20 | 3 | 5.6 | 2.1 | 4.9 |
| | 0.0 | 3 | | 2.1 | |
| 7 | 60 | 62.4 | 72.6 | 4.8 | 3.6 |
| | 0.0 | 61.8 | | 2.1 | |
| 8 | 60 | 8.2 | 10.3 | 8.7 | 8.9 |
| | 0.0 | 8.3 | | 8.4 | |
| 9 | 60 | 3.1 | 6.7 | 2.1 | 14.2 |
| | 0.0 | 3 | | 2.1 | |
| 10 | 200 | 7.9 | 7.3 | 2.7 | 7.6 |
| | 0.0 | 7.2 | | 3 | |
| 12 | 200 | 54 | 56.5 | 2.1 | 2.8 |
| | 0.0 | 60 | | 2.1 | |
| 13 | 200 | 62 | 75.8 | 2.1 | 8.7 |
| | 0.0 | 64.8 | | 2.1 | |

NOTE:
duplicate sample assay results are reported for each patient sample
Abbreviations:
FLC, free light chain;
κFLC, kappa free light chain;
λFLC. gamma free light chain;
NA, not assessed

TABLE 4
Patient demographics and baseline characteristics

| | Dose of kappamab | | | | |
|---|---|---|---|---|---|
| | 0.3 mg/kg | 1.0 mg/kg | 3.0 mg/kg | 10 mg/kg | Overall |
| Characteristic | (N = 3) | (N = 3) | (N = 3) | (N = 3) | (N = 12) |
| Median age, years (min-max) | 78 (63-83) | 56 (47-67) | 63 (52-63) | 63 (62-68) | 63 (47-83) |
| Gender, n (%) | | | | | |
| Male | 2 (67%) | 1 (33%) | 2 (67%) | 2 (67%) | 7 (58%) |
| Female | 1 (33%) | 2 (67%) | 1(33%) | 1 (33%) | 5 (42%) |
| Mean BMI, kg/m² (SD) | 22.0 (3.49) | 28.2 (4.00) | 25.4 (1.28) | 26.7 (3.48) | 25.6 (3.65) |
| Median time since diagnosis, | 93 (82-180) | 83 (64-111) | 51 (37-71) | 76 (51-191) | 79 (37-191) |
| months (min-max) | | | | | |
| Median ECOG PS (min-max) | 1 (1-2) | 0 (0-1) | 0 (0-1) | 0 (0-0) | 0 (0-2) |
| Ongoing maintenance therapy, n | | | | | |
| Thalidomide/lenalidomide (Pt #) | 2 (02, 03) | 3 (04, 05, 06) | 2 (08, 09) | 1(10) | 8 |
| Dexamethasone/prednisolone (Pt #) | 2 (02, 03) | 2 (05, 06) | 2 (08, 09) | 1(12) | 7 |
| Cyclophosphamide (Pt #) | 0 | 1 (05) | 0 | 0 | 1 |
| Median number of lines | 7 (2-8) | 6 (5-10) | 4 (3-6) | 7(6-11) | 6 (2-11) |
| of prior antineoplastic | | | | | |
| therapy^{a} (min-max) | | | | | |
| Patients with prior ASCT, n (%) | 0 (0%) | 2 (67%) | 3 (100%) | 3 (100%) | 8 (67%) |
| Overall disease response | | | | | |
| to prior antineoplastic therapy, n (%) | | | | | |
| Complete response | 0 (0%) | 1 (33%) | 0 (0%) | 0 (0%) | 1 (33%) |
| Partial response | 2 (67%) | 2 (67%) | 3 (100%) | 3 (100%) | 10 (83%) |
| Stable disease | 1 (33%) | 0 (0%) | 0 (0%) | 0 (0%) | 1 (33%) |

^{a}ASCTs were not included.
Abbreviations: ASCT, autologous stem cell transplantation BMI, body mass index; ECOG PS, European Cooperative Oncology Group performance status; N, number of patients in treatment group; n, number of patients; Pt#, patient identification number; SD, standard deviation.

TABLE 5
Summary of treatment-emergent adverse events

| Number of patients with AEs (%) [number of AEs] | | | | |
|---|---|---|---|---|
| Dose of kappamab | | | | |
| 0.3 mg/kg | 1.0 mg/kg | 3.0 mg/kg | 10 mg/kg | Overall |
| (N = 3) | (N = 3) | (N = 3) | (N = 3) | (N = 12) |

All treatment-emergent AEs

| | | | | | |
|---|---|---|---|---|---|
| Grade 1-3 | 2 (66%) [4] | 3 (100%) [3] ^{a} | 1 (33%) [2] | 2 (67%) [9] | 8 (67%) [18] |
| Grade 4/5 | 0 (0%) [0] | 0 (0%) [0] | 0 (0%) [0] | 0 (0%) [0] | 0 (0%) [0] |
| Total | 2 (67%) [4] | 3 (100%) [3] | 1 (33%) [2] | 2 (67%) [9] | 8 (67%) [18] |

Possibly, probably, or definitely related AEs

| | | | | | |
|---|---|---|---|---|---|
| Grade 1-3 | 0 (0%) [0] | 0 (0%) [0] | 0 (0%) [0] | 2 (67%) [6] | 2 (17%) [6] |
| Grade 4/5 | 0 (0%) [0] | 0 (0%) [0] | 0 (0%) [0] | 0 (0%) [0] | 0 (0%) [0] |
| Total | 0 (0%) [0] | 0 (0%) [0] | 0 (0%) [0] | 2 (67%) [6] | 2 (17%) [6] |

Possibly, probably, or definitely related AEs by System Organ Class
MedDRA Preferred Term
Gastrointestinal disorders

| | | |
|---|---|---|
| Eructation | 1 (33%) [1] | 1 (8.3%) [1] |
| Nausea | 2 (67%) [2] | 2 (8.3%) [2] |
| Total | 2 (67%) 3] | 2 (17%) [3] |

Musculoskeletal and connective tissue disorders

| | | |
|---|---|---|
| Pain in extremity | 1 (33%) [1] | 1 (8.3%) [1] |
| Total | 1 (33%) [1] | 1 (8.3%) [1] |

Respiratory, thoracic & mediastinal disorders

| | | |
|---|---|---|
| Dyspnea | 1(33%) [1] | 1 (8.3%) [1] |
| Total | 1 (33%) [1] | 1 (8.3%) [1] |

Vascular disorders

| | | | | | |
|---|---|---|---|---|---|
| Flushing | | | | 1 (33%) [1] | 1 (8.3%) [1] |
| Total | | | | 1 (33%) [1] | 1 (8.3%) 1] |
| Overall total | 0[0] | 0[0] | 0[0] | 2 (67%) [6] | 2 (17%) [6] |

^{a} One patient experienced grade 3 arthralgia.
Abbreviations: MedDRA, Medical Dictionary for Regulatory Activities; N = Number of patients studied.

TABLE 6
Summary of the pharmacokinetic parameters for
kappamab following single intravenous doses

| | Dose of kappamab | | | |
|---|---|---|---|---|
| | 0.3 mg/kg | 1.0 mg/kg | 3.0 mg/kg | 10 mg/kg |
| Parameter | (N = 3) | (N = 3) | (N = 3) | (N = 3) |
| AUC_{0-tz} | 803 | 3006 | 6864 | 22994 |
| (μg · h/mL) | (23.4) | (45.3) | (17.0) | (15.8) |
| AUC_{0-∞} | 901 | 3079 | 6927 | 23227 |
| (μg · h/mL) | (18.7) | (46.1) | (16.9) | (15.8) |
| Cₘₐₓ | 5.44 | 20.6 | 69.9 | 219 |
| (μg/mL) | (11.5) | (24.2) | (14.1) | (21.7) |
| tₘₐₓ^{a} (h) | 3.00 | 4.00 | 2.00 | 4.00 |
| | (2.00-6.00) | (3.00-4.08) | (2.00-3.00) | (3.00-6.00) |
| AUC_{0-tz}^{b} | 2678 | 3006 | 2288 | 2299 |
| (μg · h/mL) | (23.4) | (45.3) | (17.0) | (15.8) |
| AUC_{0-∞}^{b} | 3003 | 3079 | 2309 | 2323 |
| (μg · h/mL) | (18.7) | (46.1) | (16.9) | (15.8) |
| Cₘₐₓ^{b} | 18.1 | 20.6 | 23.3 | 21.9 |
| (μg · h/mL) | (11.5) | (24.2) | (14.1) | (21.7) |
| t_{1/2} (h) | 237 | 202 | 191 | 124 |
| | (24.9) | (58.1) | (10.1) | (40.3) |
| CL | 0.00555 | 0.00541 | 0.00722 | 0.00718 |
| (mL/min/kg) | (18.7) | (46.1) | (16.9) | (15.8) |
| V_{z} | 0.114 | 0.0946 | 0.120 | 0.0770 |
| (L/kg) | (41.2) | (12.3) | (27.3) | (32.5) |

Note:
data presented as geometric mean (CV %).
^{a}Data presented as median (min-max).
^{b}Data normalized for dose (mg/kg).
Abbreviations: AUC_{0-tz)}, area under the concentration versus time curve from time zero to z hours after dose; AUC_{0-∞}, area under the concentration versus time curve from time zero to infinity; CL, apparent total body clearance; Cₘₐₓ, maximum observed concentration; CV, coefficient of variation; N = number of patients studied; t_{1/2}, apparent elimination half-life; t_{max,} time of maximum observed concentration; V_{z}, apparent volume of distribution during terminal phase.

TABLE 7
Baseline measurement of serum M protein, serum kappa free light
chain (κFLC), and urine κFLC in 24-hour urine samples
at screening and at Day 1 and Day 45 after infusion with kappamab

| | Serum M protein | Serum κFLC | | | |
|---|---|---|---|---|---|
| Patient | pre-infusion | pre-infusion | Urine κFLC (mg/24 hours) | | |
| number | (g/L) | (mg/L) | Screening | Day 1 | Day 45 |
| 1 | 30 | 100 | 208 | 144 (−31%) | 107 (−49%) |
| 2 | 33 | 191 | >2604^{a} | 1902 (−27%) | >2272^{a} (−13%) |
| 3 | 15 | 118 | 1346 | 1143 (−15%) | 5964 (+343%) |
| 4 | 0 | 33.1 | 13 | 24 (+85%) | 18 (+38%) |
| 5 | 52 | 12.5 | 97 | 31 (−68%) | 103 (+6%) |
| 6 | 2 | 5.6 | 46 | 38 (−17%) | 45 (−2%) |
| 7 | 16 | 72.6 | 312 | 390 (+25%) | 286 (−8%) |
| 8 | 0 | 10.3 | 9 | 34 (+278%) | 25 (+178%) |
| 9 | 3 | 6.7 | 56 | 49 (−13%) | 39 (−30%) |
| 10 | 8 | 7.3 | 42 | 13 (−69%) | 40 (−5%) |
| 12 | 29 | 56.5 | 327 | 297 (−9%) | 363 (+11%) |
| 13 | 0 | 75.8 | 148 | 166 (+12%) | 29 (−80%) |

Note:
Values in brackets depict the percent change from baseline in urine κFLC.
^{a}Concentration exceeds upper limit of quantitation.

TABLE 8
Significance of the fixed effects of cytokine, kappamab dose,
time, and their interactions as determined by ANOVA using the
mixed-effects model to model cytokine expression levels.

| | | Statistical |
|---|---|---|
| Effects | P value | significance |
| Baseline cytokine expression^{a} | <0.001 | +++^{d} |
| Time^{b} | 0.307 | NS |
| kappamab dose^{c} | 0.581 | NS |
| Cytokine | <0.001 | +++^{d} |
| Time^{b}:kappamab dose^{c} | 0.622 | NS |
| Time^{b}:Cytokine | 0.570 | NS |
| kappamab dose^{c}:Cytokine | <0.001 | +++^{d} |
| Time^{b}:kappamab dose^{c}:Cytokine | 0.999 | NS |

Note:
Baseline cytokine expression levels (at timepoint −0.5 h before kappamab infusion) were treated as a covariate and model allowed for patient-to-patient variation by treating patients as a random effect. Analysis of deviance was assessed based on type 1 Wald's chi-squared tests.
^{a}Defined as baseline expression of each cytokine at −30 minutes before kappamab infusion.
^{b}Defined as 6, 48, 192 and 1,080 hours after kappamab infusion.
^{c}0.3 mg/kg, 1.0 mg/kg, 3.0 mg/kg, and 10 mg/kg
^{d}+ + + (P < 0.001).
Abbreviations: ANOVA, analysis of variance; h, hour; NS, not significant.

TABLE 9
Significance of change in cytokine
expression across kappamab doses.

| | | Statistical |
|---|---|---|
| Cytokine | P value | significance |
| Monokine induced by gamma | 0.002 | ++^{a} |
| interferon (MIG; CXCL9) | | |
| Interferon gamma-induced | 0.031 | +^{b} |
| protein 10 (IP10; CXCL10) | | |
| Macrophage inhibitory | 0.031 | +^{b} |
| factor (MIF) | | |
| Hepatocyte growth factor | 0.031 | +^{b} |
| (HGF) | | |
| Cutaneous T-cell-attracting | 0.037 | +^{b} |
| chemokine (CTACK, CCL27) | | |
| Granulocyte-colony | 0.037 | +^{b} |
| stimulating factor (G-CSF) | | |

Note:
P-values have been multiple-test corrected according to the false discovery rate correction procedure of Benjamini & Hochberg.
^{a}++ (P < 0.01)
^{b}+ (P < 0.05)
Abbreviations: NS, not significant.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

The present application claims priority from AU 2018904581 filed 3 Dec. 2018, the entire contents of which are incorporated herein by reference.

### REFERENCES

Asvadi et al. (2015) British Journal of Haematology, 169, 333-343.
Bates et al. (2015) J Stat Soft, 67, 1-48.
Benjamini et al. (1995) J R Stat Soc Series B Stat., 57, 289-300.
Blade et al. (1998) Br J Haematol. 102, 1115-23.
Bolomsky et al. (2016) Leukemia and Lymphoma; 57(11):2516-2525M.
Bruchfield et al. (2009) Mol Med., 15, 70-75.
Crawford et al. (2011) J Cell Commun Signal, 5, 101-110.
Giuliani et al. (2006) Haematologica., 91, 1489-97.
Hofmann K, et al. (1982) Biochemistry 21: 978-84.
Hutchinson et al. 2011, Molecular Immunology, 48, 1245-1252.
Klasen et al. (2014) J Immunol., 192, 5273-84
Kleiner et al. (2013) Mediators of inflammation; 2013:1-6(2).
Kristensen et al. (2013) Br J Haematol., 161, 373-82.
Mahtouk et al. (2010) Biochim Biophys Acta., 1806, 208-19.
Muehlinghaus et al. (2005) Blood., 105, 3965-71.
Nakayama et al. (2003) J Immunol, 170, 1136-40.
Palumbo and Anderson (2011) N Engl J Med, 364, 1046-60.
Rajkumar et al. (2011) Blood, 117, 4691-4695.
Rocci et al. (2014) Br J Haematol, 164, 841-50.
Seidel et al. (1998) Blood; 91(3):806-812 (3).
Zonder et al. (2012) Blood., 120, 552-9.

1. A method of treating multiple myeloma in a subject in need thereof, the method comprising administering to the subject an anti-KMA antibody and a proteasome inhibitor.

2. A therapeutic combination comprising a proteasome inhibitor and an anti-KMA antibody, the combination being provided for simultaneous or sequential administration.

3. The method according to claim 1 or the therapeutic combination according to claim 2, wherein the proteasome inhibitor is selected from the group consisting of marizomib, oprozomib, epoxomicin, salinosporamide A, carfilzomib, ixazomib and bortezomib.

4. The method or therapeutic combination according to claim 3, wherein the proteasome inhibitor is bortezomib.

5. The method or therapeutic combination according to any one of claims 1 to 4, wherein the anti-KMA antibody binds to or specifically binds to an epitope of KMA that is specifically bound by kappamab or that competes with kappamab for binding to KMA, wherein kappamab has a heavy chain variable region (VH) comprising a sequence set forth in SEQ ID NO: 1 and a light chain variable region (VL) comprising a sequence set forth in SEQ ID NO: 2.

6. The method or therapeutic combination according to claim 5, wherein the epitope of KMA comprises a sequence set forth in SEQ ID NO: 5.

7. The method or therapeutic combination according to any one of claims 1 to 6, wherein the anti-KMA antibody comprises a V_{H} and a V_{L}, the V_{H} comprising a complementarity determining region (CDR) 1 comprising an amino acid sequence as shown in SEQ ID NO: 6, a CDR2 comprising an amino acid sequence as shown in SEQ ID NO: 7 and a CDR3 comprising a sequence as shown in SEQ ID NO: 8 and the V_{L} comprising a CDR 1 comprising an amino acid sequence as shown in SEQ ID NO: 9, a CDR2 comprising an amino acid sequence as shown in SEQ ID NO: 10 and a CDR3 comprising a sequence as shown in SEQ ID NO: 11.

8. The method or therapeutic combination according to claim 7, wherein the V_{H} comprises an amino acid sequence at least about 95% identical to the amino acid sequence shown in SEQ ID NO: 1.

9. The method or therapeutic combination according to claim 7 or 8, wherein the V_{L} comprises an amino acid sequence at least about 95% identical to the amino acid sequence shown in SEQ ID NO: 2.

10. The method or therapeutic combination according to claim 7 or 9 wherein the V_{H} comprises an amino acid sequence as shown in SEQ ID NO: 1.

11. The method or therapeutic combination according to any one of claim 7 or 8, wherein the V_{L} comprises an amino acid sequence as shown in SEQ ID NO: 2.

12. The method or therapeutic combination according to any one of claims 1 to 6, wherein the V_{H} comprises an amino acid sequence as shown in SEQ ID NO: 1 and the V_{L} comprises an amino acid sequence as shown in SEQ ID NO: 2.

13. The method according to any one of claims 1 to 12, wherein the anti-KMA antibody is administered at a dosage ranging from about 0.3 mg/kg to 30 mg/kg.

14. The method according to any one of claims 1 to 12, wherein the anti-KMA antibody is administered at a dosage ranging from about 1 mg/kg to 10 mg/kg.

15. The method according to any one of claims 1 to 12, wherein the anti-KMA antibody is administered at about 10 mg/kg.

16. The method according to any one of claims 4 to 15, wherein the proteasome inhibitor is administered at a dose ranging from about 0.5 mg/m² to about 1.5 mg/m².

17. The method according to any one of claim 1 or 3 to 15, further comprising administering one or more additional anti-cancer agents.

18. The therapeutic combination according to claim 2, further comprising one or more additional anti-cancer agents.

19. The method according to claim 17 or the therapeutic combination according to claim 18, wherein the one or more additional anti-cancer agent(s) is/are selected from the group consisting of a chemotherapy, an immunomodulatory drug (thalidomide, lenalidomide, pomalidomide), a histone deacetylase inhibitor (panobinostat), an antibody (elotuzumab, daratumumab, isatuximab), a steroid (dexamethasone).

20. The method according to claim 17 or the therapeutic combination according to claim 19, wherein the additional anti-cancer agent is dexamethasone.

21. The method according to claim 17 or the therapeutic combination according to claim 19, wherein the additional anti-cancer agents are dexamethasone and lenalidomide.

22. The method according to any one of claim 1 or 3 to 17 or 19 to 21, wherein the anti-KMA antibody and proteasome inhibitor are administered simultaneously or sequentially.

23. The method according to any one of claim 1 or 3 to 17 or 19 to 22, wherein the anti-KMA antibody is administered monthly.

24. The method according to any one of claim 1 or 3 to 17, 19 or 22, wherein the subject has received at least one, at least two, at least three, at least four, at least five, at least six prior lines of therapy.

25. The method according to claim 24, wherein the subject achieved at least a minimal response (25% reduction in M protein) to their most recent line of therapy.

26. The method according to any one of claim 1 or 3 to 17 or 19 to 25, wherein the subject is refractory to at least one, at least two, at least three, at least four prior lines of therapy.

27. The method according to any one of claim 1 or 3 to 17 or 19 to 26, wherein the subject is refractory to at least one proteasome inhibitor.

28. The method according to claim 27, wherein the subject is refractory to bortezomib.

29. The method according to any one of claim 1 or 3 to 17 or 19 to 28, wherein the subject has relapsed myeloma.

30. The method according to any one of claim 1 or 3 to 17 or 19 to 28, wherein the subjects multiple myeloma has relapsed and is refractory to at least one proteasome inhibitor.

31. The method according to any one of claim 1 or 3 to 17 or 19 to 30, wherein the serum level of kappa free light chain in a sample obtained from the subject is less than about 250 mg/ml.

32. The method according to any one of claim 1 or 3 to 17 or 19 to 31, wherein the serum level of HGF in a sample obtained from the subject is between about 0.18 ng/ml and 1.6 ng/ml.

33. The method according to any one of claim 1 or 3 to 17 or 19 to 31, wherein the serum level of MIF in a sample obtained from the subject is between about 414 pg/ml and 4707 pg/ml.

34. The method according to any one of claim 1 or 3 to 17 or 19 to 31, wherein the serum level of CCL27 in a sample obtained from the subject is between about 150 pg/ml and 600 pg/ml.

35. The method according to any one of claim 1 or 3 to 17 or 19 to 31, wherein the serum level of G-CSF in a sample obtained from the subject is between about 20 pg/ml and 65 pg/ml.

36. The method according to any one of claim 1 or 3 to 17 or 19 to 31, wherein the serum level of CXCL9 in a sample obtained from the subject is between about 70 pg/ml and 550 pg/ml.

37. The method according to any one of claim 1 or 3 to 17 or 19 to 31, wherein the serum level of CXCL10 in a sample obtained from the subject is between about 300 pg/ml and 900 pg/ml.

38. A method of treating multiple myeloma in a subject, the method comprising selecting a subject who has high serum levels of one or more of the following factors relative to control serum levels: hepatocyte growth factor (HGF), macrophage inhibitory factor (MIF), CCL27, G-CSF, CXCL9, and CXCL10; and administering to the subject an anti-KMA antibody.

39. The method according to claim 38, wherein the serum level of HGF in a sample obtained from the subject is above about 0.5 ng/ml.

40. The method according to claim 38, wherein the serum level of MIF in a sample obtained from the subject is above about 5000 pg/ml.

41. The method according to claim 38, wherein the serum level of CCL27 in a sample obtained from the subject is above about 500 pg/ml.

42. The method according to claim 38, wherein the serum level of G-CSF in a sample obtained from the subject is above about 55 pg/ml.

43. The method according to claim 38, wherein the serum level of CXCL9 in a sample obtained from the subject is above about 550 pg/ml.

44. The method according to claim 38, wherein the serum level of CXCL10 in a sample obtained from the subject is above about 850 pg/ml.

45. The method according to any one of claims 38 to 44, further comprising administering a proteasome inhibitor.

46. The method according to claim 45, wherein the proteasome inhibitor is selected from the group consisting of marizomib, oprozomib, epoxomicin, salinosporamide A, carfilzomib, ixazomib and bortezomib.

47. The method of claim 46, wherein the proteasome inhibitor is bortezomib.

48. Use of a proteasome inhibitor and an anti-KMA antibody defined by any one of the preceding claims in the manufacture of a medicament for the treatment of multiple myeloma.

49. A proteasome inhibitor and an anti-KMA antibody defined by any one of the preceding claims for use in the treatment of multiple myeloma.
